SEQUENCE LISTING (8 sequences)

SEQ ID NO: 1 - DNA, 28 bp, synthetic construct
  source 1..28: mol_type = other DNA
actagcagctactgactagagagtgcag

SEQ ID NO: 2 - DNA, 28 bp, synthetic construct
  source 1..28: mol_type = other DNA
agtctgcagatagtagctgctgcagctg

SEQ ID NO: 3 - DNA, 29 bp, synthetic construct
  source 1..29: mol_type = other DNA
agcagtatgctagtgcgctactactatag

SEQ ID NO: 4 - DNA, 15 bp, synthetic construct
  source 1..15: mol_type = other DNA
tcgtctgtaaccatt

SEQ ID NO: 5 - DNA, 13 bp, synthetic construct
  source 1..13: mol_type = other DNA
tcgtctgtaacca

SEQ ID NO: 6 - DNA, 15 bp, synthetic construct
  source 1..15: mol_type = other DNA
atgacgtacttacgc

SEQ ID NO: 7 - DNA, 15 bp, synthetic construct
  source 1..15: mol_type = other DNA
ttgacacttagtacc

SEQ ID NO: 8 - DNA, 15 bp, synthetic construct
  source 1..15: mol_type = other DNA
ggattcgattagtag

(19) United States
(12) Patent Application Publication
(10) Pub. No.: US 2024/0309424 A1
(43) Pub. Date: Sep. 19, 2024

(54) MULTIPLEXED DETECTION OF TARGET BIOMOLECULES

(71) Applicant: PLENO, INC., San Diego, CA (US)

(72) Inventors: Angela BLUM, San Diego, CA (US); Jeffrey BRODIN, Poway, CA (US); Lorenzo BERTI, Poway, CA (US); Donald Brian EIDSON, San Diego, CA (US); Christian SCHLEGEL, San Diego, CA (US); Rachel SCHOWALTER, San Diego, CA (US); Ludovic VINCENT, San Diego, CA (US); Pieter VAN ROOYEN, San Diego, CA (US); Gavin STONE, San Diego, CA (US)

(21) Appl. No.: 18/670,364

(22) Filed: May 21, 2024

Related U.S. Application Data
(63) Continuation of application No. PCT/US2022/037781, filed on Jul. 21, 2022.
Application No. PCT/US2022/037781 is a continuation of application No. PCT/US2021/060647, filed on Nov. 23, 2021.
(60) Provisional application No. 63/307,940, filed on Feb. 8, 2022.
(60) Provisional application No. 63/346,090, filed on May 26, 2022.

(51) Int. Cl. C12Q 1/6804; C12Q 1/6811

(52) U.S. Cl. CPC C12Q 1/6804; C12Q 1/6811

(57) ABSTRACT

Methods for assaying polypeptide targets are provided that include: (a) binding each target in a sample potentially comprising a set of polypeptide targets to a capture agent to yield a set of capture agent-target complexes; (b) performing a recognition event on the capture agent-target complexes to yield capture agent-target complexes comprising an encoded probe, the encoded probe comprising a code from a set of codes, each code comprising at least one segment encoding one or more symbols that correspond to a sequence of one or more nucleotides; (c) performing a molecular transformation event to produce modified encoded probes in the presence of the target and unmodified encoded probes in the absence of the target, in which the modified probes can be amplified and the unmodified probes cannot be amplified in an amplification event; and (d) performing the amplification event and detecting the targets by decoding the codes that are amplified.

### RELATED APPLICATIONS

This application is a continuation application of International Application No. PCT/US2022/037781, filed Jul. 21, 2022, which claims the benefit of U.S. Provisional Application No. 63/346,090, filed on May 26, 2022, U.S. Provisional Application No. 63/307,940, filed on Feb. 8, 2022, and International Patent Application No. PCT/US2021/060647, filed on Nov. 23, 2021, each of which is herein incorporated by reference in its entirety.

### INCORPORATION BY REFERENCE OF SEQUENCE LISTING

The present application is being filed along with a Sequence Listing in electronic format. The Sequence Listing is provided as a file entitled 64100-717_301_SL.XML, created May 21, 2024, which is 7.84 kilobytes in size. The information in the electronic format of the Sequence Listing is incorporated by reference in its entirety.

### FIELD OF THE INVENTION

The subject matter relates to multiplexed detection of target biomolecules.

### BACKGROUND OF THE INVENTION

Molecular assays, such as immunoassays and nucleic acids assays, are commonly used to detect one or more target analytes in a sample. There is a need in the art for assays that are capable of processing and/or assaying large numbers of analytes in a multiplexed assay format.

### BRIEF DESCRIPTION OF DRAWINGS

The features and advantages of the present invention will be more clearly understood from the following description taken in conjunction with the accompanying drawings, which are not necessarily drawn to scale, and wherein:

FIG. 1 is a diagram illustrating an encoding method that uses a 4-state encoding trellis with 3 transitions per state.

FIG. 2 is a diagram illustrating an encoding trellis for a 4-bases-per-cycle pyrosequencing.

FIG. 3 is a diagram illustrating a pyro-code example, followed by a snapshot from a spreadsheet with relevant parameters. FIG. 3 discloses SEQ ID NOS 1-3, respectively, in order of appearance.

FIG. 4 shows a hypothetical emission spectrum, which is detected at varying intensities by Channel A, Channel C and Channel G, and not detected by Channel T.

FIG. 5 is a schematic diagram of an example of a coded padlock probe.

FIG. 6A is a schematic diagram illustrating an example of a process of using a surface-bound oligonucleotide to initiate an RCA reaction.

FIG. 6B is a schematic diagram illustrating an example of capturing a nanoball on a cation-coated surface.

FIG. 6C is a schematic diagram illustrating an example of capturing a nanoball on a streptavidin-coated surface.

FIG. 6D is a schematic diagram showing an example of using a biotin-streptavidin linkage to perform a surface-bound RCA reaction.

FIG. 7A is a schematic diagram of a transformation process for circularizing a linear probe to form a circular modified probe.

FIG. 7B is a schematic diagram showing RCA amplification of the circular modified probe to yield a nanoball product.

FIG. 7C is a schematic diagram showing the addition of sequencing adapters to a nanoball concatemer for subsequent clustering and sequencing.

FIG. 8 is a schematic diagram of an example of a portion of the nanoball of FIG. 7A-7C that includes restriction sites that may be used to separate repeated copies of the probe in the nanoball.

FIG. 9 is a schematic diagram of an example of a process for circularizing and amplifying unit length nanoball fragments to produce multiple RCA nanoball products.

FIG. 10 is a schematic diagram of an example of an alternative process for circularizing and amplifying unit length nanoball fragments to produce multiple RCA nanoball products.

FIG. 11 is a flow diagram of an example of a targeted analyte assay workflow.

FIG. 12 is a schematic diagram illustrating an example of a modified ELISA process of the invention.

FIG. 13A is a schematic diagram illustrating an example of a modified sandwich ELISA assay that may be used for the detection of target biomolecules in a surface-bound assay.

FIG. 13B is a schematic diagram illustrating another example of a modified sandwich ELISA assay that may be used for the detection of target biomolecules in a surface-bound assay.

FIG. 14 is a schematic diagram illustrating an example of a modified ELISA process that may be used for multiplexed detection of target biomolecules in a surface-bound assay.

FIG. 15 is a schematic diagram illustrating an example of multiplexing target detection in a surface-bound assay.

FIG. 16 is a schematic diagram illustrating an example of a solution-based assay that uses two different capture agents and a split encoded probe for the detection of a target of interest.

FIG. 17 is a schematic diagram illustrating an example of a process for detecting a target of interest using a split encoded probe integrated with the capture agents and a pair of bridging oligonucleotides to facilitate a ligation reaction to generate a readable code.

FIG. 18 is a schematic diagram illustrating an example of a process for detecting a target of interest using a split encoded probe integrated with the capture agents and a single bridging oligonucleotide to facilitate a ligation reaction to generate a readable code.

FIG. 19A is a schematic diagram illustrating an example of multiplexing target detection in a solution-based assay.

FIG. 19B is a schematic diagram illustrating another example of multiplexing target detection in a solution-based assay.

FIG. 20 is a schematic diagram illustrating some of the factors considered in the design of an encoded probe for decoding by hybridization.

FIG. 21A is a schematic diagram illustrating an overview of process for decoding by hybridization.

FIG. 21B is a schematic diagram illustrating the code space in decoding by hybridization.

FIG. 22 is a schematic diagram of an example of a method for encoding symbols onto each segment of a code.

FIG. 23 is a schematic diagram of another example of a method for encoding symbols onto a code wherein the length of the code sequence comprises a single segment that requires a relatively large number of decoding oligos.

FIG. 24 is a schematic diagram of another example of a method for encoding symbols onto a code wherein a mix of segment number and flows/segment in the decoding process balances the length of a code and the complexity required in the decoding oligo pool.

FIG. 25 is a screenshot of an example of the permutations (e.g., colors, flows/segment, total segments, and total flows) that may be used to achieve a relatively large combination space (codespace) from which to select a subset of codes.

FIG. 26A is a plot showing the relationship of the number of codes in a code space.

FIG. 26B is a summary table of the number of segments, flows, and colors required for a given number of targets for detection.

FIG. 27 is a schematic diagram of an example of a trellis code and a process of using the trellis code to select a set of codes with desired properties for an assay from a large code space.

FIG. 28A is a representation of a strategy for designing oligo segments on a probe that will encode for the symbols that make up the trellis code (or other type of code).

FIG. 28B shows examples of excluded sequences and temperature parameters for the strategy for designing oligo segments on a probe of FIG. 28A.

FIG. 29 is a representation of an overview of a decoding process comparing hard decoding vs. soft decoding. FIG. 29 discloses SEQ ID NOS 4-8, respectively, in order of appearance.

FIG. 30 is a schematic diagram of an example of a soft decoding process that may be used in the assays of the invention.

FIG. 31 is a summary of a channel model for a base calling algorithm that may be used in a soft decoding process.

FIG. 32 is a flow chart illustrating aspects of the disclosed methods.

FIG. 33 is a plot showing the quantification of the RCA nanoball product by qPCR.

FIG. 34A is a plot showing quantification of the POC assay for target protein CA-125.

FIG. 34B is a plot showing quantification of the POC assay for target protein Cyfra21-1.

FIG. 35A is a plot for the first 2 bases of a code sequence.

FIG. 35B is a panel of sequencing images for the first 2 bases of the code sequence of FIG. 35A.

FIG. 36A is a photo showing the density, size and uniformity of nanoballs generated in an RCA reaction performed on a polylysine-coated MiSeq flow cell.

FIG. 36B is a photo showing the density, size and uniformity of nanoballs generated in an RCA reaction performed on a polylysine-coated microplate.

FIG. 37A is a panel of photos of a comparison of nanoballs generated on a polylysine surface to nanoballs absorbed to a surface after an RCA solution reaction.

FIG. 37B is a pair of plots of a comparison of nanoballs generated on a polylysine surface to nanoballs absorbed to a surface after an RCA solution reaction.

### SUMMARY OF THE INVENTION

In various embodiments of the invention, a method is provided of conducting an assay for polypeptide targets, comprising: (a) binding each target in a sample potentially comprising a set of polypeptide targets to a capture agent to yield a set of capture agent-target complexes; (b) performing a recognition event on the set of capture agent-target complexes comprising use of a set of encoded probes, each encoded probe comprising a code from a set of codes, each code comprising at least one segment encoding one or more symbols that correspond to a sequence of one or more nucleotides, to yield a set of coded targets each comprising the capture agent-target complex and the encoded probe; (c) performing a molecular transformation event for each encoded probe of the set of coded targets to yield a set of modified encoded probes comprising the code in the presence of the target and unmodified encoded probes comprising the code in the absence of the target, in which the modified probes can be amplified and the unmodified probes cannot be amplified in an amplification event; and (d) performing the amplification event for each code of the set of modified encoded probes and detecting the targets by decoding the codes that are amplified.

In some instances, the set of encoded probes is a set of soft decodable probes.

In the method, the set of encoded probes may include at least 10, 100, 1,000, or 10,000 encoded probes and each of the encoded probes may include a soft decodable code.

In various embodiments, the set of encoded probes consists of tens, hundreds, thousands, or up to tens of thousands of the encoded probes wherein decoding the codes that are amplified comprises decoding the codes by a soft decoding method, and wherein the codes are trellis codes and at least a subset of the trellis codes has the same length.

In other embodiments of the invention, a method is provided for conducting an assay for polypeptide targets. The method includes: (a) binding each target in a sample that potentially includes a set of polypeptide targets to a capture agent to yield a set of capture agent-target complexes; (b) performing a recognition event on the capture agent-target complexes to yield capture agent-target complexes that include an encoded probe, the encoded probe having a code from a set of codes, each code having at least one segment encoding one or more symbols that correspond to a sequence of one or more nucleotides; (c) performing a molecular transformation event to produce modified encoded probes in the presence of the target and unmodified encoded probes in the absence of the target, in which the modified probes can be amplified and the unmodified probes cannot be amplified in an amplification event; and (d) performing the amplification event and detecting the targets by decoding the codes that are amplified.

In the methods, the set of targets may include tens of target analytes, hundreds of target analytes, thousands of target analytes, or tens of thousands of target analytes.

The set of targets may be from a sample that includes, but is not limited to, one or a combination of whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes.

The set of targets can be from a mammalian sample or a non-mammalian sample. The sample can be a plant sample, a viral sample, or a pathogen sample, and combinations thereof. In some cases, the set of targets is for pathogen detection.

The methods of the invention may include counting the decoded codes and estimating target quantity based on counts of decoded codes.

In some instances of the methods of the invention, the encoded probe is a padlock probe and the modified encoded probed is a circularized encoded probe. The capture agent may be immobilized on a surface. The capture agent-target complex may include a secondary antibody bound to the target. The encoded probe may be attached to the secondary antibody or hybridized to an oligonucleotide tag attached to the secondary antibody.

In other instances, the capture agent is two different capture agents, the encoded probe is a split encoded probe, each of the capture agents includes an oligonucleotide tag complementary to one part of the split encoded probe, and the modified encoded probed is a circularized encoded probe.

In one embodiment, the capture agent is two different capture agents, the encoded probe is a split encoded probe, each of the capture agents includes one part of the split encoded probe, the recognition event includes introduction of a pair of bridging oligonucleotides, and the modified encoded probed is a circularized encoded probe.

In another embodiment, the capture agent includes two different capture agents, the encoded probe is a split encoded probe, each of the capture agents comprises one part of the split encoded probe, the recognition event comprises introduction of a single bridging oligonucleotide, and the modified encoded probed is a linear ligated encoded probe.

In the methods of the invention, the amplification event may include a rolling circle amplification reaction to yield a nanoball that includes multiple copies of the code. The amplification event may be performed on a surface, including cases where immobilization on the surface does not include a covalent attachment to the surface. The surface may be a charged surface, including a cation-coated or anionic surface. In some cases, the cation-coated surface is a polylysine coated surface.

The encoded probes used in the assays of the invention may include one or a combination of common adapters, sequencing primers, one or more amplification primer sequences, unique identifier sequences (UMIs), restriction sites, or sample indexes.

In various embodiments of the methods of the invention, the codes that are amplified are decoded using a soft decision decoding method. For example, decoding the codes that are amplified may include recording signal produced in response to interrogation of each segment of the codes and, upon completion of the interrogation, determining a probably of the presence of each of the codes by applying a soft-decision probabilistic decoding algorithm to the recorded signal. The signal produced may include, but is not limited to, signal from one or a combination of nanopore sequencing, next-generation sequencing, massively parallel sequencing, Sanger sequencing, sequencing by synthesis (SBS), pyrosequencing, sequencing by hybridization, decoding by hybridization, single molecule real-time sequencing, SOLiD, and sequencing by ligation.

For the codes, each segment may comprise one symbol corresponding to one nucleotide. In one instance, each of the codes includes up to 50 segments for a length of each code up to 50 nucleotides. In this instance, decoding the codes that are amplified may include using sequencing by synthesis (SBS).

In other instances, each segment includes one symbol corresponding to more than one nucleotide.

In various embodiments, each code may include two or more segments, three or more segments, four or more segments, or five to sixteen segments.

In one instance, interrogation of the segments includes decoding by hybridization. At least one of the segments may be interrogated more than one time by hybridization with one or more hybridization probes each having at least one label to produce the signal. In some cases, at least four different labels may be utilized in the decoding by hybridization. The label may be an optical label or a fluorescent label.

In one example, each code includes at least four segments and at least sixteen symbols.

In the methods of the invention, the unique number of possibilities at each of the segments includes up to the number of different labels raised to the power of the number of the hybridizations per segment.

In one embodiment, at least one probe has two or more of the labels to create a pseudo label and generate a larger number of the symbols.

The length of each code from the set of codes may range from 3 to 100 nucleotides or from 3 to 75 nucleotides.

In various instances, each code from the set of codes is a predetermined code. Each code from the set of codes may be selected to avoid interaction with other assay components. Each code from the set of codes may be selected to ensure that it differs from each other code from the set of codes. Each code from the set of codes may be homopolymer free. Each code from the set of codes may be generated from a 4-ary nucleotide alphabet of A, C, G and T and generated, for example, using a 4-state encoding trellis with 3 transitions per state. In another example, each code from the set of codes is generated from a 3-ary nucleotide alphabet of a set of three of A, C, G and T and generated, for example, using a 4-state encoding trellis with 3 transitions per state.

In various embodiments, encoded probes, sets of encoded probes, and compositions including the sets of encoded probes are provided.

In one instance, a set of encoded probes is provided, each encoded probe comprising a code from a set of codes, each code is a soft decodable code comprising at least one segment encoding one or more symbols that correspond to a sequence of one or more nucleotides. The set of encoded probes may be a set of padlock probes.

The set of encoded probes may include at least 10, 100, 1,000, or 10,000 probes.

In the methods and compositions of the invention, the codes in the set of encoded probes may be the same length. In other instances, at least a subset of the set of encoded probes has codes of the same length. In some embodiments, the codes are trelliis codes.

### DETAILED DESCRIPTION OF THE INVENTION

### Definitions

“A,” “an” and “the” include their plural forms unless the context clearly dictates otherwise.

“About” means approximately, roughly, around, or in the region of. When “about” is used with a numerical range, it modifies that range by extending the boundaries above and below the numerical values indicated. “About” can modify a numerical value above and below the stated value by a variance of, e.g., 10 percent up or down (higher or lower).

“And” is used interchangeably with “or” unless expressly stated otherwise.

“Include,” “including,” and the like are to be construed in an inclusive sense as opposed to an exclusive or exhaustive sense; that is to say, in the sense of “including, but not limited to.”

“Invention,” “the invention” and the like are intended to refer to various embodiments or aspects of subject matter disclosed herein and are not intended to limit the invention to the specific embodiments or aspects of the invention referred to.

The terms “coded” and “encoded” are intended to have the same meaning and are herein used interchangeably.

“Linked” with respect to two nucleic acids means not only a fusion of a first moiety to a second moiety at the C-terminus or the N-terminus, but also includes insertion of the first moiety to the second moiety into a common nucleic acid. Thus, for example, the nucleic acid A may be linked directly to nucleic acid B such that A is adjacent to B (-A-B-), but nucleic acid A may be linked indirectly to nucleic acid B, by intervening nucleotide or nucleotide sequence C between A and B (e.g., -A-C-B- or -B-C-A-). The term “linked” is intended to encompass these various possibilities.

“Optimum,” “optimal,” “optimize” and the like are not intended to limit the invention to the absolute optimum state of the aspect or characteristic being optimized but will include improved but less than optimum states.

“Sample” means a source of target or analyte. Examples of samples include biological samples, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Samples may be from any organism (e.g., prokaryotes, eukaryotes, plants, animals, humans) or other sample (e.g., environmental or forensic samples).

“Set” includes sets of one or more elements or objects. A “subset” of a set includes any number elements or objects from the set, from one up to all of the elements of the set.

“Subject” includes any plant or animal, including without limitation, humans.

“Target” means a target polypeptide or epitope or a proxy for the target analyte of interest (e.g., an antibody conjugated with oligonucleotide). “Target” with respect to a polypeptide includes wild-type and mutated polypeptides of any length, including proteins and peptides.”

“Decoding” with respect to a code includes determining the presence of a known code or a probability of the presence of a known code with or without determining the sequence of the code. Decoding may be hard decision decoding. Decoding may be soft decision decoding.

“Identify,” “determine” and the like with respect to codes, targets or anlaytes of the invention are intended to include any or all of: (A) an indication of the presence or absence of the relevant code, target or analyte, (B) an indication of the probability of the presence or absence of the relevant code, target or analyte, and/or (C) quantification of the relevant code, target or analyte.

“Hard decision decoding” or “hard decision” refers to a method or model that includes making a call for each nucleotide in a nucleic acid segment (commonly referred to as a “base call”) in order to determine the sequence of nucleotides in the nucleic acid segment. Models of the invention incorporate hard decision decoding models. The particular nucleic acid being decoded may be or include a code of the invention.

“Soft decision decoding” or “soft decision” refers to a method or a model that uses data collected during a sequencing or decoding process to calculate a probability that a particular nucleic acid or nucleic acid segment is present. The probability may optionally be calculated without making a base call for each nucleotide in a nucleic acid segment. In another example, a probability is calculated without making a hard call that a string of nucleic acids in a segment are present. Instead of making a hard call for each nucleotide or nucleotide segment, a probabilistic decoding algorithm is applied to the recorded signal upon completion of signal collection. A probability of the presence of each of the codes may be determined without discarding signal in contrast to hard decision decoding method in which hard calls are made during the signal collection process. In soft decision decoding, the data may, for example, include or be calculated from, intensity readings in spectral bands for signals produced by the sequencing/decoding chemistry. In one embodiment, soft decision decoding uses data collected during a sequencing/decoding process to calculate a probability that a particular nucleic acid segment from a known set of sequences is present. Models of the invention may be used for soft decision decoding. The particular nucleic acid or nucleic acid segment being decoded may be or include a code of the invention.

“Phasing” or “signal phasing” means misalignment of SBS cycles during an SBS process caused by the non-incorporation of a nucleotide during a cycle or by the incorporation of two or more nucleotides during an SBS cycle.

“Droop” or “signal droop” means signal decay that occurs during an SBS process, which may be caused by some complementary strands being synthesized as part of the SBS process being blocked, preventing further nucleotide incorporation.

“Sample” means a set of nucleic acids for testing. A sample preparation process may be used to produce a sequencing-ready sample from a raw sample or partially processed sample. Note that one or more samples may be combined for sample preparation and/or sequencing and may be distinguished post-sequencing using sample-specific DNA barcodes linked to sample fragments.

“Crosstalk” refers to the situation in which a signal from one nucleotide addition reaction may be picked up by multiple channels (referred to as “color crosstalk”) or the situation in which a signal from a nanoball or sequencing cluster interferes with an adjacent or nearby cluster or nanoball (referred to as “cluster crosstalk” or “nanoball crosstalk”).

“Color channel” means a set of optical elements for sensing and recording an electromagnetic signal from a sequencing reaction. Examples of optical elements include lenses, filters, mirrors, and cameras.

“Spectral band” or “spectral region” means a continuous wavelength range in the electromagnetic spectrum.

Headings are included herein for reference and to aid in locating the various sections. These headings are not intended to limit the scope of the concepts described with respect to the headings.

The description and examples should not be construed as limiting the scope of the invention to the embodiments and examples described herein, but as encompassing all modifications and alternatives falling within the true scope and spirit of the invention.

### Encoded Assays

The disclosure provides encoded assays for detection of target analytes in a sample. At a high level, in an encoded assay, a target analyte (“target”) is detected based on association of the target with a code and detection of the code is a surrogate for detection of the analyte.

In various embodiments, an encoded assay may include a recognition event in which a target is uniquely recognized by a recognition element. The recognition event may be effected by submitting targets of a set of targets to a recognition event, in which each target is uniquely recognized by and bound to a recognition element associated with a code, thereby yielding a set of coded targets comprising the target and the recognition element.

In various embodiments, an encoded assay may include a transformation event, in which a high-fidelity molecular transformation of the recognition element associated with a code produces a modified recognition element. The transformation event may be effected by submitting each recognition element of the set of coded targets to a transformation event, in which a molecular transformation of each recognition element produces a modified recognition element, thereby yielding a set of modified recognition elements comprising the code.

In various embodiments, an encoded assay may include a decoding event, which detects the code as a surrogate for detection of the analyte, e.g., by decoding the code (and optionally decoding other elements). The decoding event may include an amplification step in which each code of the set of modified recognition elements is amplified, thereby yielding a set of amplified codes. Amplified codes of the set of amplified codes may have their sequences decoded using a variety of techniques, including for example, microarray detection, or nucleic acid sequencing. In some cases, the detection step may be integrated with the amplification step, e.g., as in amplification with intercalating dyes.

In one embodiment, the method may include:
(i) submitting each target of a set of targets to a recognition event, in which each target is uniquely recognized by and bound to a recognition element associated with a code, thereby yielding a set of coded targets comprising the target and the recognition element;
(ii) submitting each recognition element of the set of coded targets to a transformation event, in which a molecular transformation of each recognition element produces a modified recognition element, thereby yielding a set of modified recognition elements comprising the code;
(iii) submitting each code of the set of modified recognition elements to an amplifying event, in which each code is amplified, thereby yielding a set of amplified codes;
(iv) submitting each amplified code of the set of amplified codes to a decoding event.

In one embodiment, the method may include:
(i) a recognition event in which the target is uniquely recognized by a recognition element, which associates a code (and optionally other elements) with the target via the recognition element;
(ii) a transformation event, in which a high-fidelity molecular transformation of the recognition element produces a modified recognition element that produces a readable code;
(iii) a decoding event, which decodes the code as a surrogate for detection of the analyte.

As described in more detail herein, the recognition event, transformation event, and the decoding event may occur sequentially, or combinations of the steps may occur simultaneously, e.g., as a single combined step. For example, the transformation event and the coding event may be simultaneous, such that the sequential process involves (i) recognition event, followed by (ii) transformation event/coding event, followed by (iii) decoding event.

To further illustrate the encoded assays:
(i) In the recognition event, the target may be detected by a targeted molecular binding event, such as binding of the target by a complementary sequence or a polypeptide binder.
(ii) In the transformation event, a ligation or a gap-fill ligation may produce the modified recognition element, i.e., a version of the recognition element that is ligated or gap-fill ligated.
(iii) In the coding event, a code reagent may be associated with the modified recognition element based on recognition of the modified recognition element. For example, the novel coded padlock probes of the invention may be configured with a sequence that recognizes the modified recognition element and circularize only if the modified recognition element is present.
(iv) In the decoding event, the decoding of the code may involve any means of determining the presence of the code (and optionally other elements).

The codes may be error corrected and thus easy to distinguish from each other, so they can be detected a low abundance and in the presence of high level of background and in the presence of many other codes.

Since many assays can be converted into codes, the invention provides for multiomic assays where a sample is analyzed in multiple parallel workflows that are analyte-dependent and then converge codes that can be then decoded simultaneously in a single platform. Parallel assay workflows may be merged into a single workflow, where multiple targets and target-types (e.g., nucleic acids and polypeptides) may be decoded simultaneously in a single workflow and also read simultaneously within the same readout platform.

Following recognition and transformation, the codes may be decoded and matched to targets for identification and/or quantification of targets present in the sample.

### Code Design and Decode

The encoded assays of the invention make use of codewords or codes. The codes may be decoded as surrogates in the place of direct analysis of target analytes. As an example, a target analyte may be a particular nucleic acid fragment (e.g., a nucleic acid fragment with a specific mutation); in the assays of the invention, a codeword may be associated with the nucleic acid fragment and the codeword may be read to identify the presence of the nucleic acid fragment in the sample.

For example, a code may in some embodiments be a predetermined sequence ranging from about 3 to about 100 or more nucleotides or about 3 to about 75 nucleotides. Codes may have sequences selected to avoid inadvertent interaction with other assay components, such as targets, probes, or primers. Code sequences may be selected to ensure that codes differ from each other to permit unique identifiability during the decoding process.

The invention includes a dataset or database of codes generated using the methods of the invention. The dataset or database may associate the codes with other assay elements, such as primers or probes linked to the probes. The invention also includes a method of making a probe set comprising synthesizing probes having the sequences set forth in the dataset or database.

### Homopolymer-Free Encoding

In one embodiment, the codes are homopolymer-free codes. For standard genomic applications that use a full 4-ary nucleotide alphabet of {ACGT}, the method uses a 4-state encoding trellis with 3 transitions per state.

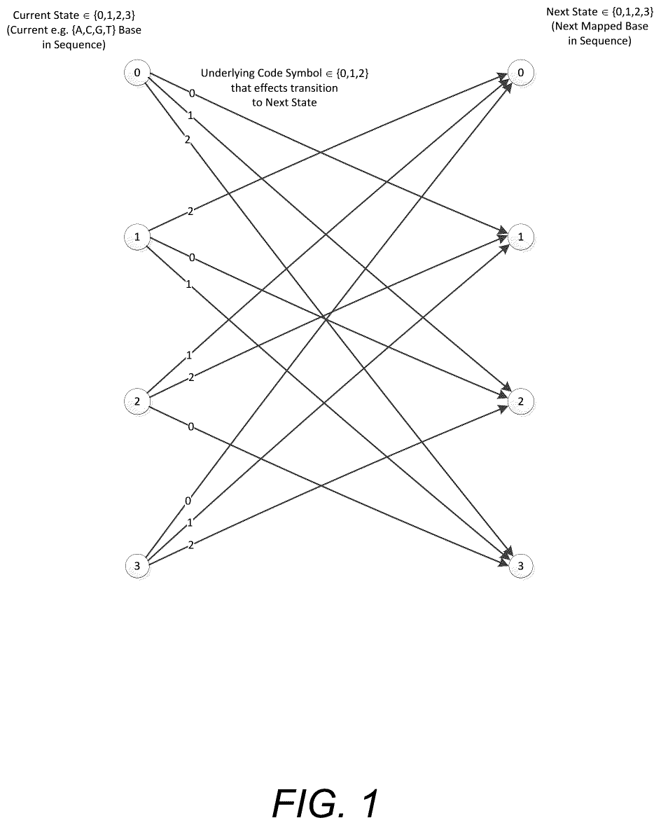

As illustrated in FIG. 1, the current state is the last mapped nucleotide, and the next state is the next (to-be) mapped nucleotide. By forbidding a transition from the current state (say, the ‘A’ state) in the present trellis section (of 4 states), to the analogous same state (of ‘A’) in the next trellis section (of 4 states), a repeated mapping to the same nucleotide base—in any generated sequence—is avoided. An ‘A’ state can only transition to a ‘C’, ‘G’, or ‘T’ state in the next trellis section. Since this involves 3 transitions per state, the mapping trellis is mated to an underlying 3-ary (i.e., ternary-) alphabet error correction code that drives transitions through trellis sections. The underlying (ternary) error correction code is the mechanism that guarantees all generated codewords differ in multiple sequence positions.

A similar method may apply to 3-ary alphabets (where only 3 of the four nucleotide bases, say {CGT} are used), and 5-ary or higher alphabets, where the underlying correction code uses an alphabet of order one less than the mapping alphabet.

In one embodiment, codes for the set of codes are selected using a 4-ary alphabet, avoid homopolymers, and every code in the set is different from every other code in the set. The codes may be generated using the trellis method.

In one embodiment, codes for the set of codes are selected using a 3-ary alphabet, avoid homopolymers, and every code in the set is different from every other code in the set. The codes may be generated using the trellis method.
(i) In another embodiment, a homopolymer-free code composed from a 4-ary nucleotide alphabet of {ACGT} may be generated as follows:
(ii) From GF(4) (i.e., the quaternary algebraic alphabet), select an error correction code that will deliver many more codewords than necessary (because some of the generated codewords will later be eliminated);
(iii) Generate all of the codewords for the code;
(iv) Assess the number of repeated symbol locations in each codeword;
(v) Re-order the list of codewords, sorting by the number of base-repeat instances in each codeword.
(vi) From the re-ordered sort, keep only the top K codewords, where K is the desired library size of codewords (this will eliminate the codes with the highest number of polymer-repeats; each repeat will require subsequent fixing that weakens the overall code.)
(vii) For each codeword in the list of survivors, ‘smart fix’ the repeat positions in each codeword with the following procedure:
   a. Start from the beginning base position in a codeword, and find the first repeat instance of a base;
   b. Go to the second base in the first repeat instance, its base assignment will require change;
   c. If the second base is not at the end of a codeword, look ahead one base position in the codeword, and assess the assignment there;
   d. For the second base (in the repeat), choose a new base assignment that is also different from the base assigned one sample ahead; n that, in addition to removing a length-2 run, this step will also fix a length-3 run;
   e. Process the revised codeword at each remaining repeat location, fixing the second base in each repeat using the process outlined in steps c-d.

This method will eliminate all repeats. The same method can be applied to generate homopolymer codes for 3-ary alphabets (eg., {C, G, T}), and larger 5-ary+alphabets (such as oligopolymers).

Codes may be optimized for pyrosequencing and similar cyclic serial dispensation schemes. In one embodiment, the invention provides a locus code-encoding approach for pyrosequencing or similar serial (rather than pooled) primer dispensation methods. The method generates homopolymer-free codes.

When the locus code is encapsulated between header and tail bases, all generated codewords finish decoding at the same time. The technique avoids unexpected spurious incorporations that change how long in time that a codeword needs to finish its decoding. This is important because then a sequencer only need sample for a prescribed number of samples to obtain complete data for decoding the samples, regardless of the underlying codeword. This also keeps all codewords candidates aligned, so that the theoretical design distances between codewords are maintained.

The aforesaid synchrony ensures that soft decision block decoding techniques can be applied during the decoding of its blocks of samples. This soft decision decoding guarantees that SNR requirements are improved by at least 2 dB—and sometimes by many factors-more when the signal strength significantly fades during the reception of codeword samples.

In pyrosequencing, nucleotides are dispensed sequentially (and non-overlappingly) in a cycle, such as G, C, T, A, G, C, T, A, G, C, . . . etc. This encoding is quite original because it doesn't directly encode bases; instead, it encodes base positions within G, C, T, A cycles. Each cycle element can be either populated, or unpopulated—and multiple elements within a cycle can be populated. For this to be implemented, the underlying code must be derived from a binary alphabet, with 1s and 0s. To emphasize, with these codes, more than one base can be incorporated within a single G, C, T, A dispensation cycle. This also implies that sequencing, though serial in nature, can be fast. And with the underlying {0, 1} alphabet that underpins and drives the encoding of the populated/unpopulated cycle positions, all codewords are guaranteed to be of the same length—and to finish decoding in the same amount of time.

To provide coding gain, the sequence of 0s and 1s that comprise each codeword are derived from constructions of optimal binary error correction codes. Such codes possess many redundant parity bits, and these parity bits are designed such that each codeword varies from each other in multiple positions. This quality results in strong error correction capabilities.

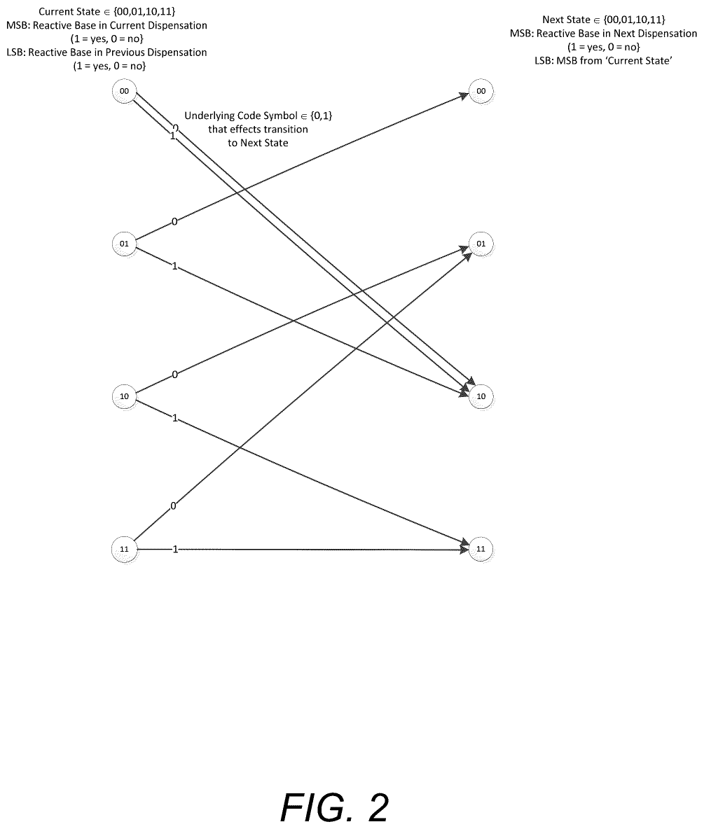

FIG. 2 illustrates an encoding trellis for a 4-bases-per-cycle pyrosequencing. The techniques may be used for encoding 3-cycle, 3-base-alphabet, and 5+-cycle, 5-and-higher-alphabet oligo-polymer hybrid schemes.

Note the use of 4 states in the trellis. Each state represents previous mappings of that last two positions:
(i) both unpopulated, (00);
(ii) both populated, (11);
(iii) newest-populated and older-unpopulated, (10);
(iv) newest-unpopulated and the older populated, (01).

Transitions to next states indicate an update which either does not populate or does populate the next position in a sequence.

Four (4) states are used to correctly implement a pyrosequencing scheme that is homopolymer-free; one position is populated every 3 positions. Note that if 3 consecutive positions were allowed to be unfilled, then the 4^{th} position would need to be filled (because an unzipped hybrid will have an opening to at least one of the four nucleotides). That 4^{th} position being filled would result in generation of a homopolymer (repeat) of bases in a sequence—since the last filled base was the same base in the cycle before.

This aforementioned restriction explains the double transition from the 00 state to the 10 state in the trellis diagram. A current state of 00 transitioning to a next state of 00 would imply 3 positions in a row were unfilled.

Optimal error correction codes are constructed to maximize distance between their sets of codewords. They are not constrained to disallow runs of three consecutive zeros. That would reduce the degrees of freedom they use to maximize distance. By contrast, the mappings to pyro-sequenced positions comply with homopolymer-free and pyrosequencing constraints.

All other transitions in the picture design trellis are natural results of populating a position with a ‘0’ or a ‘1’ and updating the next state to reflect that transition. Since 7 of the 8 transitions in the trellis perfectly express the underlying error correction code's structure, such a code can be quite effective and powerful.

Weakening transitions occur when the underlying code has 3 consecutive zeros. One way to reduce those appearances is to use the sorting methodology described above. This method modestly reduces the library of codes. This method also ensures that the pyro-mapped codewords that best reflect the underlying binary code's structure are faithfully reproduced, while those least reflective are not.

Another method to improve the weakening due to transitions involves breaking up strings of zeros by interleaving the code. Within a code, the (systematic) information section of bits—which precede the redundant section of parity bits—are the bits where the most consecutive zeros are usually seen. One way to eliminate those strings of zeros is to interleave the entire code design, so that the parity and information bits are intermingled. All codewords may be intermingled by the same interleaving pattern. The interleaving technique does not help for the all-zeros codeword, which is generated by almost all linear codes. The all-zeros codeword can be excluded from the codeword set.

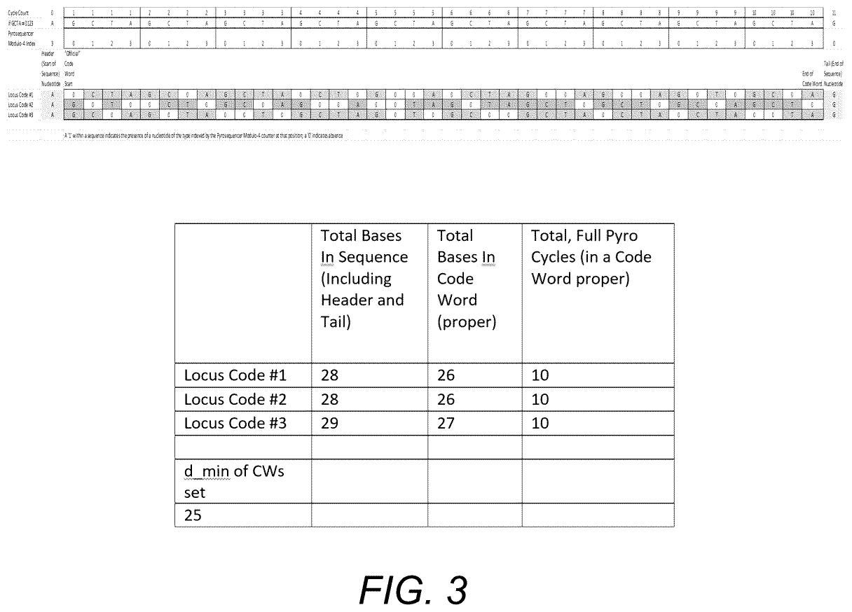

FIG. 3 shows a pyro-code example, followed by a snapshot from a spreadsheet with relevant parameters. The code is a 10-cycle, 40 position code that maps {GCTA} in cycles. It possesses a huge minimum distance between codewords, and is an example code accommodating three codewords. Note that the number of bases assigned to each codeword is not the same, although, clearly, from the illustration, all codewords are of the same time duration, and would finish decoding at the same time. Observe also the usage of populated ‘header’ and ‘tail’ positions. These are used to encapsulate the codeword and ensure that it is homopolymer free throughout. These terminating positions may be butted-up against the ends of the codewords for effective encapsulation.

For the purposes of the specification and claims, the codes of the invention that are based on an encoding trellis as illustrated in FIG. 1, FIG. 2, and FIG. 3 can be referred to herein as “trellis codes”.

### Amplifying and Reading Codes

In an encoded assay, a target is detected based on association of the target with a code, and detection of the code is used as a surrogate for detection of the analyte. A variety of techniques may be used to amplify and read the codes.

In one embodiment, codes of the invention are amplified using rolling circle amplification (RCA) to produce DNA nanoballs that include many duplicates of the code. An RCA reaction may include one or more rounds of amplification to produce the nanoball product. A nanoball may be from about 10,000 to about 1,000,000 nucleotides in length. A nanoball may include from about 100 to about 10,000 copies of the amplified code.

In one embodiment, the codes of the invention are amplified using a linear PCR amplification reaction to generate double stranded DNA amplicon products.

In one embodiment, codes of the invention are amplified using bridge amplification to produce clusters of oligos on a surface.

In one embodiment, codes of the invention are amplified on bead surfaces to produce bead-attached oligos.

In one embodiment, the amplified codes are read in a sequencing reaction.

In one embodiment, codes of the invention are detected using a patterned array, such as a microarray comprising oligos which are complimentary to the codes.

In one embodiment, codes of the invention are detected in situ, i.e., in a cell or tissue.

In one embodiment, in situ detection comprises decoding the code in situ.

In one embodiment, codes of the invention are detected using an electronic/electrical sensing mechanism.

A variety of techniques and models may be used to identify a nucleic acid code of the invention. In one embodiment, the invention provides models that make use of hard decision decoding methods or models. In another embodiment, the invention provides models that make use of soft decision decoding methods or models.

When using soft decision decoding techniques, it is not necessary for the model to identify each base specifically. For example, signals generated during each nucleotide addition cycle of a sequencing process may be detected and recorded to produce a data set that may be used as input into a model of the invention to calculate a probability that a specific code is present without requiring a hard decoding model. Although it is not necessary in a soft decision decoding model to make a hard decision about the identity of each nucleotide, a model developed according to the methods of the invention may nevertheless include a model for assigning a probability or identity to each nucleotide in the sequence of a code.

Data gathered during a sequencing process may, for example, include intensity readings for signals produced by the sequencing chemistry in various spectral bands. For example, in some cases the data is collected across a set of spectral bands that corresponds to part or all of the spectral bands expected to be produced by a series of nucleotide extension steps during a sequencing process.

In some embodiments, it is not necessary to filter light from each nucleotide extension step in order to distinguish between the nucleotides. Instead, a set of intensity readings may be detected, stored and used as input into a model of the invention for determining a probability that a particular code is present. In other embodiments, one or more filters may be used to refine signals from a sequencing process.

A model may be developed or trained using sequencing data from known codes, such as signal intensity data across a predetermined spectrum, during a sequencing process. The model may be used to calculate a set of probabilities across a set of one or more codes, indicating, for example, for each code, a probability that it is present in a sample.

In some cases, the model is developed or trained using data corresponding to color intensity signals across multiple color channels. In some cases, the model is developed or trained using data corresponding to color intensity signals across four color channels, each generally corresponding to the signal produced by addition of one of the four nucleotides A, T, C or G during a sequencing process. As discussed elsewhere in this specification, the channels may experience color crosstalk.

A model may be built using data obtained using multiple light sensing channels. Each channel may be specific for a specific frequency bandwidth. In some cases, the model may be built using four channels, wherein the bandwidth of each channel may be selected for signals produced by addition of one of the four nucleotides A, T, C or G. In other cases, more or less than four channels may be used to collect data used to produce the model.

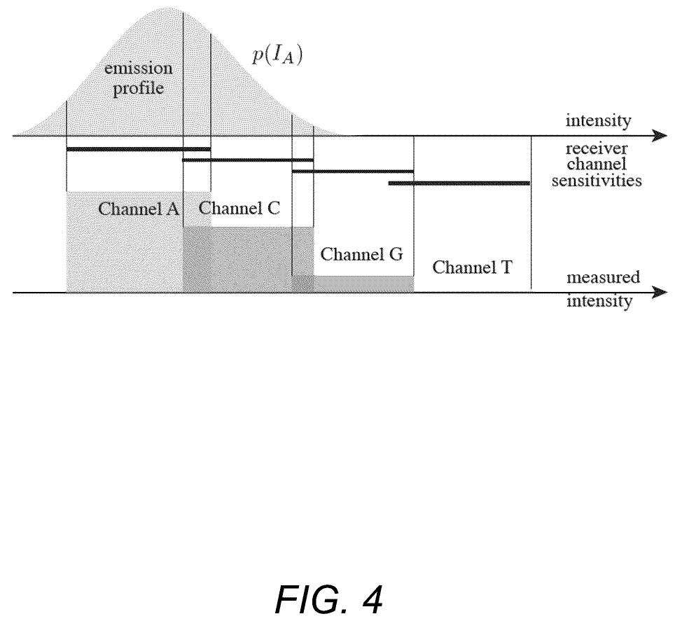

In certain embodiments of the invention, each channel detects a bandwidth region of a fluorescence signal produced by addition of one of the four nucleotides. Nevertheless, the bandwidth of the signal produced by addition of one of the four nucleotides may be spread across a spectral band that overlaps with other channels. This effect is illustrated in FIG. 4. FIG. 4 shows a hypothetical emission spectrum, which is detected at varying intensities by Channel A, Channel C and Channel D, and not detected by Channel T.

As will be discussed in the examples below, a color crosstalk model may be empirically developed and used as input into the model of the invention for producing a probability that a code is present. Relative coefficient strength may be experimentally determined across color channels for signal produced by addition of each nucleotide (A, T, C, G) from empirically produced test data.

Other factors that may be included in a statistical model according to the invention for calculating a probability that a code is present include signal phasing, signal droop, color cross-talk values, fluctuations in in color cross-talk values, noise, amplitude noise, gaussian amplitude models, and base calling algorithms.

The model of the invention may also take into account various sources of noise and error, such as variability in the concentration of the active molecules in the assay, variability in color channel response due primarily to limited ability to estimate the color channel responses individually for each cluster, and background and random error noise sources. A concentration noise model may be used to model the variable density of active molecules for a given cluster. A transduction noise model may be included to model variability in the color crosstalk matrix.

Accurately modeling the biochemical opto-mechanical processes in DNA sequencing is a complex process. Furthermore, to derive the inputs for a soft decision probabilistic signal estimator requires estimating the parameters driving the model, as well as having strong confidence that the model is accurate. Under these two assumptions, metrics can be computed that work directly with the received signals. In the commercially available base call algorithms, channel distortion effects are compensated for before the decision process; however, in soft decision decoding of the invention it is not necessary to compensate for distortions before decoding. Embodiments which do not compensate for distortions before decoding will have the advantage of avoiding information loss compensations, such as inversions.

The probability that a particular code is present may be indicative of the probability that a particular target associated with the probe is present. Data indicating the probability that a particular target is present may be used, for example, to calculate probabilities relevant to diagnosis or screening of various medical conditions, or selection of drugs for treatment of various medical conditions.

The disclosure provides encoded probes that can be decoding using soft decision decoding methods or models. The codes may be generated using the trellis method and the codes may be referred to as “trellis codes”. The probes of the invention may be padlock probes that include a soft decodable code, such as a trellis code. The probes of the invention may be a dual probe that includes a soft decodable code, such as a trellis code.

The disclosure provides assays that make use of encoded probes that may be decoded using soft decision decoding (“soft decoding”). In various embodiments, the assays make use of mixtures of probes, each with a soft decodable code. A mixture may include 100s, 1000s, or 10000s of encoded probes.

In some instances of the methods of the invention, determining the sequence of the code or the presence of the code is performed without making a specific base call for each nucleotide in the code.

In some embodiments, a hybridization-based detection method may be used to determine the code. In one embodiment, the amplified codes are determined using oligonucleotide probes in a hybridization-based reaction. The amplified codes may be determined using sequencing by hybridization. In one example, the hybridization-based detection method uses fluorescently labeled oligonucleotide probes. The code data may then be used as a digital count of the target-specific decoding events.

### Multiplexed Detection of Target Biomolecules

The disclosure provides assays for multiplexed detection of target analytes. In various embodiments, the assays provide a readout that can be measured alongside the readout of other molecular assays, such as nucleic acid assays, that may be performed in parallel, thereby enabling a multiomic platform for the analysis of different analytes in a sample. The target may be any biomolecule (e.g., carbohydrate, protein, nucleic acid, or small molecule) for which there is a set of orthogonal affinity tags.

The assays make use of a set of capturing agents for capturing targets. The assays make use of a set of encoded oligonucleotide probe sequences (“encoded probes”) for detecting a panel of targets. The encoded probes may be used as surrogates for bioanalysis of the targeted biomolecules.

An assay using encoded probes (i.e., an encoded assay) may include: (i) a capture event, in which a target is uniquely recognized and bound by a capture agent to form a capture agent-target complex; (ii) a recognition event, in which the capture agent-target complex is uniquely recognized by a recognition element associated with a code (i.e., an encoded probe); (iii) a transformation event, in which a molecular transformation of the recognition element produces a modified recognition element comprising the code; and (iv) a decoding event, that uses the code as a surrogate for detection of the target analyte, e.g., by recognizing or determining the presence or sequence of the code (and optionally other elements).

An encoded assay may be a solution-based assay.

An encoded assay may be a surface-bound assay.

An encoded assay may be a hybrid assay that includes a surface-bound component and a solution-based component.

An encoded assay maybe performed in a plate-based format (e.g., a multi-well plate). The multi-well plate may include, for example, an array of nanowells.

An encoded assay may be performed on a microfluidics device.

The encoded probe may include other functional sequences such as sequencing primers, one or more amplification primer sequences, unique identifier sequences (UMIs) and sample indexes. The sequencing primers may, in some cases, be adjacent to the code sequence. The amplification primer sequences may, in some cases, be universal primer sequences that are common to all probes in a set of encoded probes. The unique identifier sequences (UMIs) and sample indexes may also be soft decodable codes, such as trellis codes.

An encoded probe may be a padlock probe that includes a recognition element associated with a code. The code may be a soft decodable code, such as a trellis code.

Thus, for example, the disclosure provides a padlock probe in which the terminal sequences comprise a probe and a soft decodable code is provided between the terminal sequences. Similarly, the disclosure provides a padlock probe in which the terminal sequences comprise a probe and a trellis code is provided between the terminal sequences. The disclosure provides a set of 10 or more padlock probes in each of which (A) the terminal sequences comprise a probe and (B) a soft decodable code is provided between the terminal sequences. The disclosure provides a set of 100 or more padlock probes in each of which (A) the terminal sequences comprise a probe and (B) a soft decodable code is provided between the terminal sequences. The disclosure provides a set of 1000 or more padlock probes in each of which (A) the terminal sequences comprise a probe and (B) a soft decodable code is provided between the terminal sequences. The disclosure provides a set of 10,000 or more padlock probes in each of which (A) the terminal sequences comprise a probe and (B) a soft decodable code is provided between the terminal sequences. In certain embodiments, the foregoing sets are provided in the absence of any padlock probes that do not include the soft decodable codes. In certain embodiments, the foregoing sets are provided with codes that are homopolymer-free and soft decodable.

The disclosure provides a set of 10 or more padlock probes in each of which (A) the terminal sequences comprise a probe and (B) a trellis code is provided between the terminal sequences. The disclosure provides a set of 100 or more padlock probes in each of which (A) the terminal sequences comprise a probe and (B) a trellis code is provided between the terminal sequences. The disclosure provides a set of 1000 or more padlock probes in each of which (A) the terminal sequences comprise a probe and (B) a trellis code is provided between the terminal sequences. The disclosure provides a set of 10,000 or more padlock probes in each of which (A) the terminal sequences comprise a probe and (B) a trellis code is provided between the terminal sequences. In certain embodiments, the foregoing sets are provided in the absence of any padlock probes that do not include the trellis codes. In certain embodiments, the foregoing sets are provided with codes that are homopolymer-free trellis codes.

An encoded probe may be a molecular inversion probe that includes a recognition element associated with a code. The code may be a soft decodable code, such as a trellis code.

The disclosure provides a set of 10 or more molecular inversion probes in each of which (A) the terminal sequences comprise a probe and (B) a soft decodable code is provided between the terminal sequences. The disclosure provides a set of 100 or more molecular inversion probes in each of which (A) the terminal sequences comprise a probe and (B) a soft decodable code is provided between the terminal sequences. The disclosure provides a set of 1000 or more molecular inversion probes in each of which (A) the terminal sequences comprise a probe and (B) a soft decodable code is provided between the terminal sequences. The disclosure provides a set of 10,000 or more molecular inversion probes in each of which (A) the terminal sequences comprise a probe and (B) a soft decodable code is provided between the terminal sequences. In certain embodiments, the foregoing sets are provided in the absence of any molecular inversion probes that do not include the soft decodable codes. In certain embodiments, the foregoing sets are provided with codes that are homopolymer-free and soft decodable.

The disclosure provides a set of 10 or more molecular inversion probes in each of which(A) the terminal sequences comprise a probe and (B) a trellis code is provided between the terminal sequences. The disclosure provides a set of 100 or more molecular inversion probes in each of which(A) the terminal sequences comprise a probe and (B) a trellis code is provided between the terminal sequences. The disclosure provides a set of 1000 or more molecular inversion probes in each of which (A) the terminal sequences comprise a probe and (B) a trellis code is provided between the terminal sequences. The disclosure provides a set of 10,000 or more molecular inversion probes in each of which (A) the terminal sequences comprise a probe and (B) a trellis code is provided between the terminal sequences. In certain embodiments, the foregoing sets are provided in the absence of any molecular inversion probes that do not include the trellis codes. In certain embodiments, the foregoing sets are provided with codes that are homopolymer-free trellis codes.

The transformation event may include a ligation or gap-fill ligation reaction to produce the modified recognition element comprising the code.

The decoding event may include an amplification step in which the code sequence (among other elements) is amplified. Amplification may be by any method of amplification, including for example, on-surface PCR, isothermal amplification, rolling circle amplification, and/or ultrarapid amplification. Surface based amplification may be performed using PCR with surface-anchored primers (e.g., Illumina bridge amplification technology) or recombinase polymerase amplification (RPA) (e.g., ExAmp technology).

In one embodiment, the amplification step comprises a rolling circle amplification (RCA) reaction to generate a nanoball product.

In one embodiment, an encoded probe may include a sequence which may prevent RCA of the probe, thereby allowing for linear double-stranded PCR products. The non-extendable sequence may, for example, be located between a pair of amplification primer sequences.

In one embodiment, an encoded probe may include a restriction enzyme site that may be cleaved to yield a linear DNA molecule.

In some embodiments, the amplified code may be sequenced to determine the sequence of the code associated with the target. Any sequencing technology may be used to sequence. Examples of sequencing technologies that may be used include sequencing by synthesis (e.g., pyrosequencing; sequencing by reversible terminator chemistry (Illumina)), avidity sequencing (Element Biosciences), sequencing by hybridization, sequencing by ligation, and nanopore sequencing.

In some embodiments, a sequencing library may be generated from a set of modified recognition elements comprising the codes. The library may be sequenced to determine the code associated with a target of interest. The code data may then be used as a digital count of the target-specific decoding events.

In one embodiment, a sequencing library comprising the code (among other elements) may be generated from a circularized padlock probe.

In one embodiment, a sequence library comprising the code (among other elements) may be generated from a nanoball product.

In one embodiment, a nanoball or a portion of the nanoball that includes the code (and optionally other elements) may be directly sequenced to determine the code associated with the target of interest. The code data may then be used as a digital count of the target-specific decoding events.

The nanoball product may be loaded onto a substrate surface and the sequence of the code associated with the captured target determined. In one example, the substrate surface is a well of a multi-well plate. In another example, the substrate surface is a sequencing flow cell.

In one embodiment, the sequencing substrate surface includes a cation layer coating, and the negatively charged nanoballs bind to the substrate surface. The nanoball may then be sequenced to decode the code associated with the captured target.

In one embodiment, the nanoball product may be bound to a sequencing substrate surface via a streptavidin-biotin linkage.

In one embodiment, the nanoball product may be bound to a sequencing substrate surface via hybridization to oligonucleotides immobilized on the substrate surface.

In one embodiment, the nanoball output product may be used to generate a sequencing library that may be sequenced to decode the code associated with the captured target.

In one embodiment, the code sequence may be isolated and then sequenced to decode the code associated with the captured target. In one example, the code may be sequenced using nanopore sequencing technologies.

In some embodiments, the capturing agent may be an antibody or fragment thereof that is specific for a target biomolecule of interest. In one example, the antibody (or fragment thereof) may, for example, be used in an immuno-detection process, such as a modified enzyme-linked immunosorbent assay (ELISA).

In some embodiments, the capturing agent may be an aptamer (e.g., an oligonucleotide or peptide sequence) that is specific for a target biomolecule of interest, wherein binding of the capture agent with the target of interest forms an aptamer-target complex. In one embodiment, the aptamer or a sequence related to the aptamer (e.g., an extension of the aptamer) may be read as a code.

In some embodiments, the capturing agent may be a synthetic (or semi-synthetic) molecule such as one that includes unnatural amino acids or bases.

The number of capture agents (e.g., antibodies) may be increased or decreased depending on the desired specificity of the assay.

In various embodiments, an oligonucleotide that is complementary to an encoded probe may be used to “tag” a capture agent-target complex for subsequent binding to the encoded probe. The oligonucleotide tag bound to the detection antibody may be used to link the specificity of the capture antibody to the code readout.

In some embodiments, the oligonucleotide tag may include an amplification primer sequence. In one example, the primer sequence may be used to initiate an RCA reaction.

In some embodiments, an oligonucleotide tag may be conjugated to the capture agent (e.g., an antibody). The oligonucleotide tag may, for example, be conjugated to the capture agent via a covalent or noncovalent bond. The linkage between the oligonucleotide tag and the capture agent may be cleavable or non-cleavable.

In some embodiments, two or more oligonucleotide tag sequences may be conjugated to a capture agent and used to generate multiple nanoball output products, thereby increasing assay signal output.

In some embodiments, the oligonucleotide tag may be a self-readable code. For example, the oligonucleotide tag may be a circularizable sequence that can be amplified and readout as a code.

A target biomolecule may be any biomolecule for which a capture agent or a set of independent (orthogonal) capture agents and encoded oligonucleotide sequences (e.g., “affinity tags”) may be assigned.

In some embodiments, the target of interest may be a protein. In one example, the protein target may be an epithelial cell marker such as the Cyfra 21-1 fragment of cytokeratin 19. In another example the protein target may be a glycosylated protein, such as the CA-125 protein shed by epithelial cells that may be detected in a blood sample (e.g., a serum sample).

In some embodiments, the target of interest may be a carbohydrate. In some embodiments, the target of interest may be a nucleic acid (or fragment thereof). In some embodiments, the target of interest may be a small molecule.

### Coded Padlock Probes

The disclosure provides assays that make use of novel padlock probes comprising codes that may be used as a surrogate for detection of a target, e.g., by recognizing or determining the presence or sequence of the code (and optionally other elements). The code in a padlock probe may be a soft decodable code (e.g., a trellis code). A coded padlock probe may include target-specific regions that may be used for target recognition and enrichment. A coded padlock probe may include a 5′ terminal phosphate that may be used to facilitate ligation (i.e., circularization) after target recognition. A coded padlock probe may include a 3′ nucleotide that is the complement to a nucleotide at a target site of interest (e.g., a 3′ SNP-specific nucleotide). A coded padlock probe may include an RCA priming site that includes a primer sequence suitable for priming an RCA reaction. A coded padlock probe may include a soft-decodable code.

For example, the coded padlock probe may include regions at the 3′ and 5′ ends that are complementary to regions of a target. The probe regions may hybridize to the target, and the probe may be circularized, e.g., by a ligation or gap-fill ligation reaction. As described elsewhere in this disclosure, the target may be a nucleic acid analyte (e.g., mRNA, cfDNA etc.) or a proxy for the analyte of interest (e.g., an antibody conjugated with oligonucleotide).

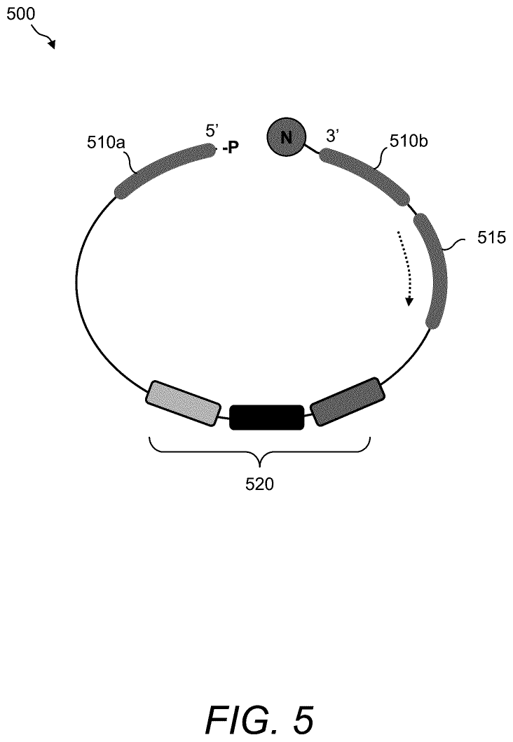

FIG. 5 is a schematic diagram of an example of a coded padlock probe 500. Coded padlock probe 500 may include a 5′ target specific region 510a and a 3′ target specific region 510b that are complementary to regions of a target (not shown). The target may be a nucleic acid analyte (e.g., mRNA, cfDNA, etc.) or a proxy for the analyte of interest (e.g., an antibody conjugated with an oligonucleotide). Target specific region 510a may include a 5′ terminal phosphate (P) that may be used to facilitate ligation (i.e., circularization) after target recognition. Target specific region 510b may include one or more terminal 3′ nucleotides “N” complementary to a nucleotide at a target site of interest. In this example, the 3′ target site specific nucleotide “N” may be a SNP specific nucleotide.

Target specific regions 510a and 510b may hybridize to the target, and the probe may be circularized. For example, when the complementary nucleotide is present in the target, the 3′ SNP specific nucleotide hybridizes to the target, enabling circularization, e.g., by ligation or gap-fill ligation. Other types of features or mutations may be detected by varying the terminal nucleotide (N) or nucleotides of target specific region 510a and/or target specific regions 510b to hybridize when the target feature is present and not hybridize when the target feature is not present.

Coded padlock probe 500 may include an RCA priming site 515 that includes a primer sequence suitable for priming an RCA reaction. In this example, RCA priming site 515 is downstream from target specific region 515b. However, other locations are possible, as long as the positioning the primer site doesn't interfere with the other functions of the probe, e.g., the probe hybridization function and the encoding function.

A coded padlock probe may optionally include other functional sequences. For example, the probe may include index sequences which are unique oligo identifiers present in the probe sequence or inserted as part of the assay. Index sequences, such as sample barcodes, allow differentiation among different samples, experiments, etc. during the decoding event (i.e., reading (decoding) the code).

The coded padlock probe may include unique molecular identifiers. UMIs may be inserted anywhere within the probe to address downstream readout and data analysis purposes. For example, UMIs may be introduced to distinguish unique recognition events with single-molecule resolution during the readout. UMI's may facilitate error correction and/or individual molecule counting.

A coded padlock probe may include other primers in addition to the priming region required for RCA amplification. Other priming regions may, for example, be present to facilitate the readout of an index, a UMI or other oligonucleotide sequences present in the probe. Priming regions may allow parallel or serial reading schemes. They may also be used to increase the amount of multiplexing or allow sequential readout. For instance, if a plurality of probes or amplified objects are present, only those containing a specific primer will be amplified or read. Primers may also be used to facilitate the capture and immobilization of a probe or amplified object onto a surface (e.g., via DNA-DNA hybridization).

A coded padlock probe may include one or more sequences recognizable by enzymes, such as endonucleases. Various sequences may be selected and used to facilitate additional transformations, such as digestion, nick or gap formation, phosphorylation etc. In one embodiment, the probe includes one or more restriction sites.

A coded padlock probe may include one or more non-natural NTP components. Examples include phosphorothioate groups, locked DNA (LNA), peptide DNA (PNA) and others, which may be included to improve certain features of the probe, such as melting temperature for target recognition, or primer recognition, or resistance to degradation. Additionally, abasic NTPs (“wobble bases”) may be included in the probe sequence to add degeneracy to targeting or priming regions and extend the ability to recognize a broader number of complementary sequences.

A coded padlock probe may include one or more chemical moieties. Such chemical moieties may be included in the probe structure or added at any stage of the workflow to enable additional transformations or properties. Examples include cleavable groups to open or linearize the probe, reactive groups to add additional components such as dyes, and groups to facilitate immobilization on surfaces.

A coded padlock probe may include CRISPR recognition sequences, oligo sequences designed to be recognized by CRISPR enzymes and replaced with other arbitrary sequences. The probe may optionally include one or more oligo sequences designed to be recognized by transposases and replaced with other arbitrary sequences.

A coded padlock probe may optionally include one or more adapter primers for compatibility with sequencing by synthesis (SBS) and other non-SBS platforms. The adapter primers may be included in the probe sequence or added at any stage as part of the workflow. Such adapter primers may be used directly to immobilize, cluster, extend, and amplify as precursor activities to a decoding run by SBS.

In one embodiment, a padlock probe assay workflow may include:
(i) hybridizing the probe to a capture agent-target complex;
(ii) optionally, extending the hybridized probe to fill any single-stranded gap remaining between the two probe arms;
(iii) circularizing the probe when the capture agent-target complex is present;
(iv) cleaning-up (e.g., by exonuclease or other mean) non-circularized probes remaining after ligation;
(v) amplifying the circularized probe by RCA or other methods;
(vi) capturing of the amplified product on a surface;
(vii) degrading the amplified product to generate a sequencing compatible library;
(viii) preparing the library for sequencing, using sequencing sample preparation workflows suitable for a desired sequencing platform; and reading out or decoding the code.

### Index Sequences

Index sequences, such as sample barcodes, allow differentiation among different samples, experiments, etc. during the decoding event. Indexes may be added to a padlock probe using a variety of strategies.

Indexes may be added during the synthesis of a padlock probe. In this case, for every probe manufactured, the number of probes is N×P, where N is the number of indices and P is the plexity of the probe pool.

Indexes may be added after probe synthesis as part of manufacturing or at a site of use as a step prior to performing an encoded assay. In this case, only one synthesis is required for each probe and additional functional elements. Additional functional elements may be added to a probe to enable insertion of an index. Examples of functional elements that may be added include (i) non-natural nucleotides (e.g., biotin, amine, etc.) and (ii) polynucleotides that enable biochemical transformation of the probe to contain an index sequence such as adapters for ligations or extension ligations, restriction endonuclease recognition sites, and transposome binding sites.

Indexes may be added during an encoded assay. For example, a ligation reaction to insert an index can occur at the same time as ligation of the padlock probe at the target site of interest to generate a circularized padlock probe (i.e., the transformation event). In some cases, the ligation reaction may be a gap-fill extension/ligation reaction.

Indexes may be added after ligation of the padlock probe and RCA by including modified nucleotides during the RCA reaction. The modified nucleotides may then be coupled to an index sequence. In cases where there is a covalent or non-covalent interaction, either moiety can be linked to the index sequence or incorporated during RCA.

Examples of coupling strategies include: (i) ligand protein pairs such as biotin-streptavidin, antigen-antibody, CLIP tag and SNAP tag pair (i.e., O6-benzylguanine derivatives coupling to O6-alkylguanine-DNA-alkyltransferase, wherein either the protein or the substrate may be bound to the probe), carbohydrate-protein pairs (e.g., lectins), and digoxigenin-DIG-binding protein; (ii) peptide-protein pairs (e.g., SpyTag-SpyCatcher); and (iii) hybridizing indexes to a common sequence on the RCA product.

Indexes may be added to RCA products by restriction endonuclease cleavage followed by index ligation.

Indexes may be added to RCA products using a transposase enzyme that fragments and indexes the RCA products.

### Surface Attachment

The encoded assays of the invention may be performed on a surface. For example, a target may be immobilized on a surface for conducting assays of the invention. The probes of the invention may be immobilized on a surface for conducting assays of the invention. DNA nanoballs of the invention may be immobilized on a surface for conducting assays of the invention. Various intermediate assemblies of molecules of the assays of the invention may be immobilized on a surface for conducting assays of the invention.

Various steps of the invention may be performed on a surface, such as target capture, recognition events, transformation events, amplification, and/or decoding events, i.e., determination of the absence or presence of the code (e.g., by sequencing or hybridization-based detection).

Thus, for example, the disclosure provides a surface having a probe as described herein immobilized on the surface. The disclosure provides a surface having a nanoball as described herein immobilized on the surface. The disclosure provides a surface having a target immobilized on the surface. The disclosure provides a surface having a target immobilized on the surface with a probe as described herein hybridized to the target. The disclosure provides a surface having a probe immobilized on the surface with a target as described herein hybridized to the probe. The disclosure provides a surface having a target nucleic acid immobilized on the surface, and a protein or peptide bound to the target nucleic acid. The disclosure provides a surface having a target nucleic acid immobilized on the surface, and an antibody, aptamer, binder, or antibody fragment bound to the target nucleic acid. The disclosure provides a surface having a ligand that has affinity for any of the foregoing immobilized on the surface. For example, the ligand may have affinity for a probe as described herein, a nanoball as described herein, or a target as described herein. The ligand may, for example, be a protein, peptide, antibody, aptamer, binder, or antibody fragment.

A variety of surfaces may be used for the surface attachments described herein. In various embodiments, the surface includes an oxide, a nitride, a metal, an organic or an inorganic polymer (e.g., hydrogel, resin, plastic or other).

The surface may take a variety of forms, e.g., it may be flat or curved. It may be beads or particles. In some cases, the surface is the surface of a flow cell. Beads or other particles may in some embodiments range in size from less than 100 nm up to several centimeters.

Various surface modifications may be used to permit attachment of various components of the assays of the invention to a surface. For example, various anchoring ligands may be used (e.g., streptavidin, biotin, aptamers, antibodies, etc.). Chemical handles, such as click chemistry handles, may be used. Examples include azides, alkynes, unsaturated bonds, amines, carboxylic acids, NHS, DBCO, BCN, tetrazine, epoxy and the like. Single-or double-stranded oligonucleotides may be used. Size ranges of the oligonucleotides may, in some cases, be from about 10 to about 200 nucleotides. Proteins or peptides may be used for surface attachment. Charge-based molecules or polymers may be used, e.g., polyethylenimine.

Various techniques may be used to prepare a surface for binding to a target or to a component of an assay of the invention. In one example, a flow cell with primers may be used. A splint DNA segment that comprises a segment complementary to the primer and a segment that is complementary to the target or the component of the assay may be hybridized to the primer. A variety of splints may be used on a surface, with various subsets of the splints having different segments complementary to different components of the invention or different targets. Specific splints may be arranged on different regions of a surface. For example, splints may be arranged in a manner that permits the identification of distinct regions of a surface targeted to specific analytes or components of the assays.

In various embodiments, amplification of a nucleic acid may occur on the surface. The nucleic acid may be a target or any nucleic acid component of an assay of the invention. For example, a target analyte may be amplified on a surface, or a probe of the invention may be amplified on a surface, and/or a fragment of any of the foregoing may be amplified on a surface. The amplification may be performed on a bead or particle, or on a flat surface, such as on the surface of a flow cell.

It should also be noted that DNA may be amplified in solution, e.g., in an aqueous suspension or emulsion, such as in microdroplets. Solution-based amplification may be performed, for example, in an open environment, such as the well of the microtiter plate, in a nanowell, or in an enclosed space, droplet in an emulsion, or on a flow cell or other microfluidic device.

Amplification may be by any method of amplification, including for example, PCR, isothermal amplification and/or ultrarapid amplification.

Attachment for immobilization of components of the assays or of targets may be covalent or non-covalent (e.g., Coulombic in nature), temporary or permanent, and/or rendered labile when subject to a particular stimulus.

Examples of mechanisms of lability include:
Enzymatic—protease, restriction endonuclease, CRISPR-Cas9
Chemical—reduction, hydrolysis, nucleophilic attack, displacement, reducing of a disulfide bond
Temperature—melting of duplexed hybridized DNA, thermodynamically unfavorable conditions (Positive deltaG)
pH—hydrazone, carbonate, etc.
Light—O-nitrobenzyl or derivatives where absorption of light of a particular wavelength(s) can cause bond rearrangements or cleavage. Light sensitive groups include nitro-benzene derivatives
Ligand mediated—competitive competition for binding site (see examples below)
   Peptide-tagged oligos with protein interactions—e.g., Spy-catcher. The moiety may be the ligand or the protein.
   Peptide-tagged oligo with heavy metal interactions—e.g., Hexa-histidine—to Cu. The moiety may be the ligand or the protein.
   CLIP tag and SNAP tag pair—i.e., O6-benzylguanine derivatives coupling to O6-alkylguanine-DNA-alkyltransferase. Either the protein or the substrate may be bound to the oligo.
   Carbohydrate-protein pairs, e.g., lectins
   The moiety may be a ligand (e.g., biotin, digoxigenin) coupled to a fluorescently-tagged protein (e.g., avidin, streptavidin, DIG-binding protein)
Cleavage can be performed by cleaving a moiety dangling on a nucleotide, or a nucleotide or a nucleobase within the oligo sequence or the di-nucleotide linkage, e.g., uracil and USER cocktail (uracil-N-deglycosylase (UNG)) followed by Endonuclease VIII or FPG (Formamidopyrimidine DNA Glycosylase with Bifunctional DNA glycosylase with DNA N-glycosylase and AP lyase activities)
Cleavage can be performed by an enzyme

### Surface-Based Workflows

A variety of surface-based workflows are possible within the scope of the assays disclosed. In some embodiments, a surface-based workflow may use a padlock probe that includes a recognition element associated with a code. The code may be a soft decodable code, such as a trellis code. In some embodiments, a surface-based workflow may use a dual probe that includes a recognition element associated with a code (e.g., a trellis code).

In some embodiments, a surface-based workflow may include immobilizing a target on a surface and hybridizing a probe to the target. In one embodiment, a surface-based workflow may include:
(i) immobilizing the target on a surface;
(ii) hybridizing a probe to the immobilized target;
(iii) circularizing the probe to produce a circular modified probe; and
(iv) releasing the circular modified probe from the target.

A target may be immobilized on a surface by a capture agent bound to the surface. In some embodiments, the capture agent may be a pair of antibodies (or a fragments thereof) and binding of the antibodies to the target forms an immobilized capture agent-target complex.

In some embodiments, a first antibody in the pair of antibodies may be immobilized on the surface and the second antibody may be provided in a solution.

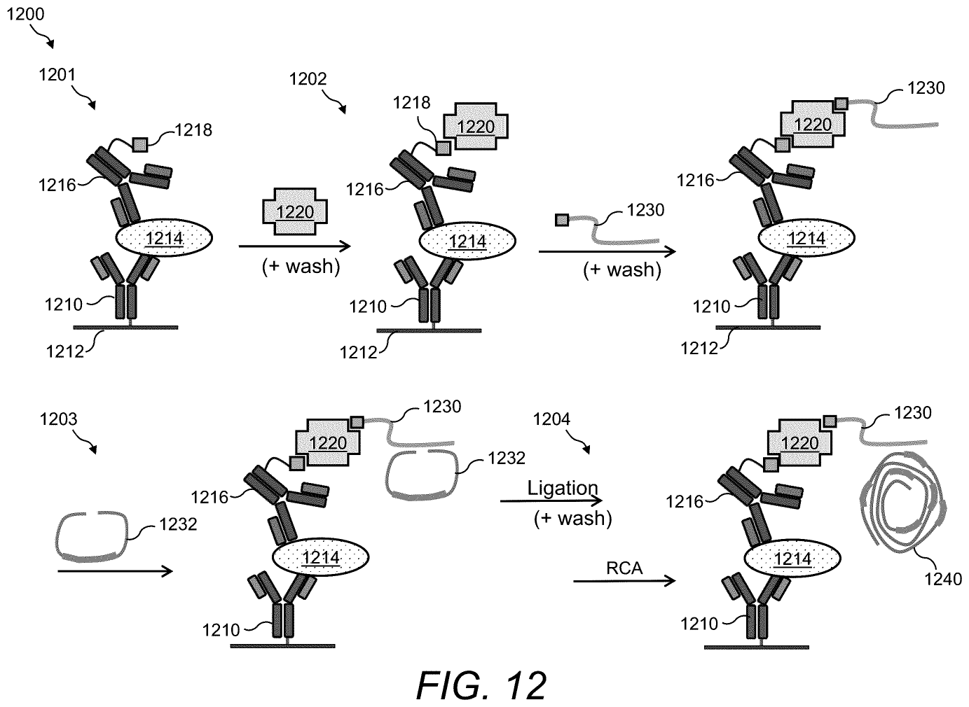

In one embodiment, an intermediary agent may be used to mediate hybridization of the probe to the immobilized capture agent-target complex. An example of using an intermediary agent to mediate hybridization of the probe to the capture agent-target complex is described in more detail hereinbelow with reference to FIG. 12.

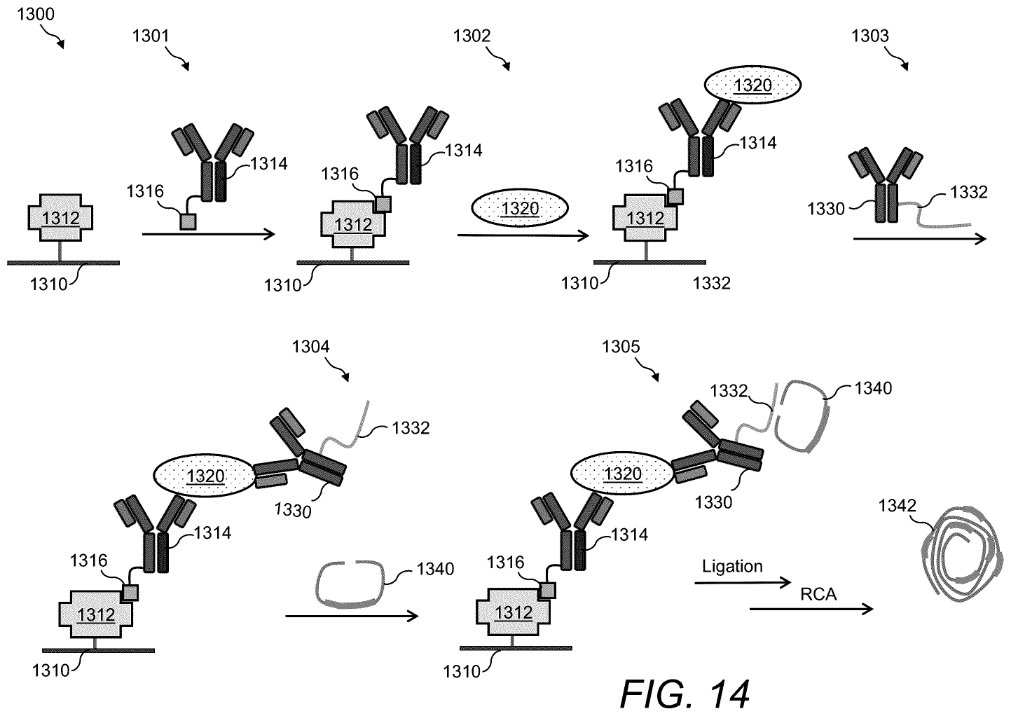

In one embodiment, an antibody in the pair of antibodies may be modified with an oligonucleotide tag sequence that may be used to mediate hybridization of the probe to the immobilized capture agent-target complex. An example of using an antibody conjugated to an oligonucleotide tag (“antibody tag”) to mediate hybridization of the probe to the immobilized capture agent-target complex is described in more detail hereinbelow with reference to FIG. 14.

In some cases, the RCA reaction may be performed in a solution that remains in contact with the surface on which the target is immobilized (e.g., in the same container, well, reservoir, liquid volume or droplet). In some cases, the solution comprising the released modified probe may be transferred to a separate container prior to performing the RCA reaction. In some cases, the solution comprising the released modified probe may be transferred to a different surface prior to performing the RCA reaction.

In some embodiments, the circular modified probe is not released from the immobilized capture-agent complex. In this case, the oligonucleotide tag conjugated to the capture agent (e.g., an antibody in a pair of antibodies) may be used to prime an RCA reaction to generate a nanoball product.

### Amplification Strategies

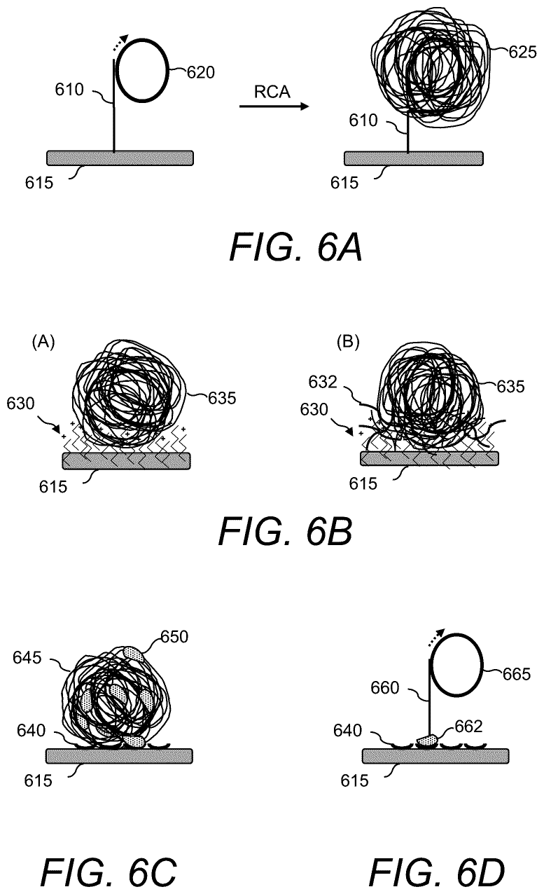

Rolling circle amplification (RCA) may be used to produce nanoballs as part of the assays of the invention. An RCA reaction may be performed as a surface-bound reaction. For example, RCA may be initiated by an oligonucleotide bound to a surface (e.g., beads, flow cells, microwell, or nanowells). Any method may be used to bind the oligonucleotide to the surface. In one example, the oligonucleotide may be covalently bound to the surface. FIG. 6A is a schematic diagram illustrating an example of a process of using a surface-bound oligonucleotide to initiate an RCA reaction (indicated by the arrow). An oligonucleotide 610 may be covalently attached to a surface 615. Oligonucleotide 610 may include an RCA primer sequence that is complementary to an RCA primer site on a probe 620. Oligonucleotide 610 may be used to capture probe 620 by hybridization of the complementary sequences and initiate the RCA reaction. Because oligonucleotide 610 is covalently bound to the surface, the surface-bound RCA reaction generates a nanoball 625 that is covalently attached to the surface.

In another example, a cation-coated or anionic surface (e.g., beads, flow cells, microwells, or nanowells) may be used to capture nanoballs. In one example, the cation-coated surface may be a polylysine-coated surface. FIG. 6B is a schematic diagram illustrating an example of capturing a nanoball on a cation-coated surface. A surface 615 may be coated with a polylysine coating 630. An RCA reaction may be performed in the presence of the polylysine coated surface, resulting in simultaneous immobilization and amplification of a nanoball 635. RCA primers may be supplied in solution (panel A) or bound to the polylysine-coated surface prior to performing the RCA reaction (panel B).

In another example, a streptavidin-coated surface (e.g., beads, flow cells, microwells, or nanowells) may be used to capture nanoballs. In this approach, biotin-linked deoxynucleotides may be incorporated into the nanoballs during RCA. The nanoballs will then be bound to the surface by a biotin-streptavidin linkage. FIG. 6C is a schematic diagram illustrating an example of capturing a nanoball on a streptavidin-coated surface. A surface 615 may be coated with a streptavidin coating 640. An RCA reaction may be performed in the presence of the streptavidin coated surface using biotin-linked deoxynucleotides to produce a nanoball 645 that includes biotin moieties 650 resulting in simultaneous immobilization and amplification of nanoball 645.

In another embodiment, biotin linked RCA primers may be bound to a surface by a streptavidin-biotin linkage and used to initiate an RCA reaction as described above with reference to FIG. 6A. An example of using a biotin-streptavidin linkage to perform a surface-bound RCA reaction is shown in FIG. 6D. A surface 615 may be coated with a streptavidin coating 640. An oligonucleotide 660 that includes a biotin moiety 662 may be attached to surface 615 through a biotin-streptavidin linkage. Oligonucleotide 616 may include an RCA primer sequence that is complementary to an RCA primer site on a probe 665. Oligonucleotide 660 may be used to capture probe 665 by hybridization of the complementary sequences and initiate the RCA reaction (indicated by the arrow) to produce a nanoball. Amplification in the presence of the streptavidin coated surface further anchors nanoball to the surface.

Following the formation of a nanoball, a determination may be made with respect to the identity of the code. Prior to making the determination, various secondary processing steps are possible within the scope of the assays described herein. The probe may include various elements that facilitate secondary processing steps. Examples include restriction endonuclease sites and CRISPR sites.

The nanoball may be converted to double-stranded DNA (dsDNA) prior to fragmentation. The dsDNA nanoball may be fragmented. In one embodiment, the probe includes restriction sites which are replicated in the nanoball, and the nanoball is fragmented using a restriction enzyme having specificity for the restriction sites.

CRISPR may be used to fragment the nanoball at specific sites.

Random fragmentation of nanoballs may be performed, using known fragmentation techniques.

Tagmentation may be performed on the nanoball, and the tagmentation may be used to add sequencing adapters.

### Sequencing Preparation

This disclosure provides a variety of techniques for amplifying and preparing circularized probes for sequencing. In certain embodiments, amplification and preparation for sequencing may be performed sequentially (e.g., PCR+ primer ligation). In certain embodiments, amplification and preparation for sequencing may be performed in a single reaction (e.g., adapter addition via PCR). Addition of sequencing adapters may be performed with or without RCA amplification of circularized probes.

In one embodiment, sequencing adapters are added via PCR. In this case, amplification and preparation for sequencing may be a single step. Depending on the probe design, the code, UMI, and index may be read in a single step or in two separate reads with a dehybridization step.

In one embodiment, RCA products (nanoballs) may be fragmented with restriction endonucleases (RE) to yield a multitude of code-containing single stranded nucleic acids. The single-stranded nucleic acids (i.e., the RE reaction products) may then be prepared for sequencing by ligation to adapter sequences.

In one embodiment, sequencing adapters may be added by transposomes that simultaneously fragment double-stranded DNA and add adapters.

As discussed elsewhere in the application, the assays of the invention include a transformation step. Typically, the transformation involves circularization of a probe when a target is present (e.g., by ligation or gap-fill ligation).

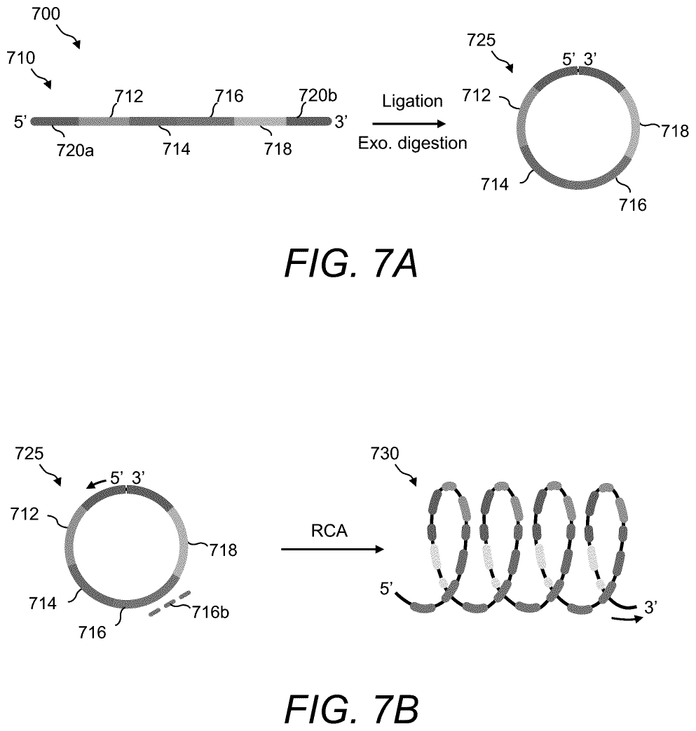

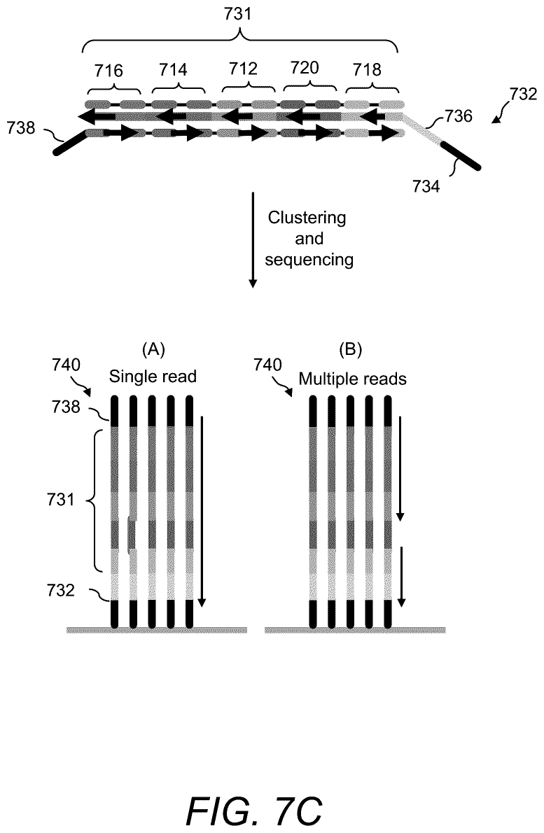

FIG. 7A is a schematic diagram of a transformation process 700 for circularizing a linear probe to form a circular modified probe. In this example, a probe 710 includes a UMI sequence 1512, a code 714, an SBS primer 716, and an index primer 718 all situated between a 5′ target recognition element 720a and a 3′ target recognition element 720b. In the presence of a target (not shown), probe 710 is circularized in a ligation reaction to yield a circular modified probe 725. The ligation reaction may be followed by an exonuclease digestion step to remove unligated probes 710 and target.

The circular modified probe shown in FIG. 7A may, in some cases, be amplified in a rolling circle amplification to form a nanoball product. FIG. 7B is a schematic diagram showing RCA amplification of the circular modified probe to yield a nanoball product. For example, in an RCA reaction an SBS primer 716b that is the reverse complement to SBS primer 716 may be hybridized to circular modified probe 725 and used to initiate the RCA reaction to generate a nanoball 730. Nanoball 730 is a polymeric molecule (concatemer) that includes multiple repeated copies of circular modified probe 725, wherein each copy includes SBS primer 716, code 714, UMI sequence 712, target recognition elements 720, and index primer 718. In this example, the complement (i.e., copy) of modified probe 725 is indicated by the dashed line.

In some embodiments, the RCA products (nanoballs) may be sequenced directly. In some embodiments, sequencing adapters may be added by PCR amplification, followed by clustering and sequencing.

FIG. 7C is a schematic diagram showing the addition of sequencing adapters to a nanoball concatemer for subsequent clustering and sequencing. The PCR reaction may use a pair of amplification primers 732 and 738. Amplification primer 732 may include a sequencing adapter sequence 734 (e.g., a P7 adapter sequence) and an index sequence 736 (e.g., a sample index sequence). Amplification primer 738 may include a second sequencing adapter sequence (e.g., a P5 adapter sequence). Amplification primers 732 and 738 are used in the PCR reaction to initiate amplification of nanoball 730 to generate multiple single probe copies 740 of modified probe 725 that now include the adapter sequences and the index sequence. In this example, a single probe copy 731 (indicated by the dashed lines) of the sequences in the original circular modified probe 725 is shown. A bridge amplification reaction may then be performed to generate a clonal cluster 740 for sequencing. Sequencing may be performed as a single read (A) or as multiple reads (B). Sequencing as a single read provides the UMI sequence, the code sequence, and the index sequence. Sequencing as multiple reads may include, for example, one read to provide the UMI and code sequences, and a second read to provide the index sequence.

In another embodiment, the probes of the invention may include restriction sites. The probes may be designed with restriction sites, or the restriction sites may be added to the probes as part of the assay process. The restriction sites will be amplified into the nanoball and will provide multiple sites at which to cut the nanoball into fragments.

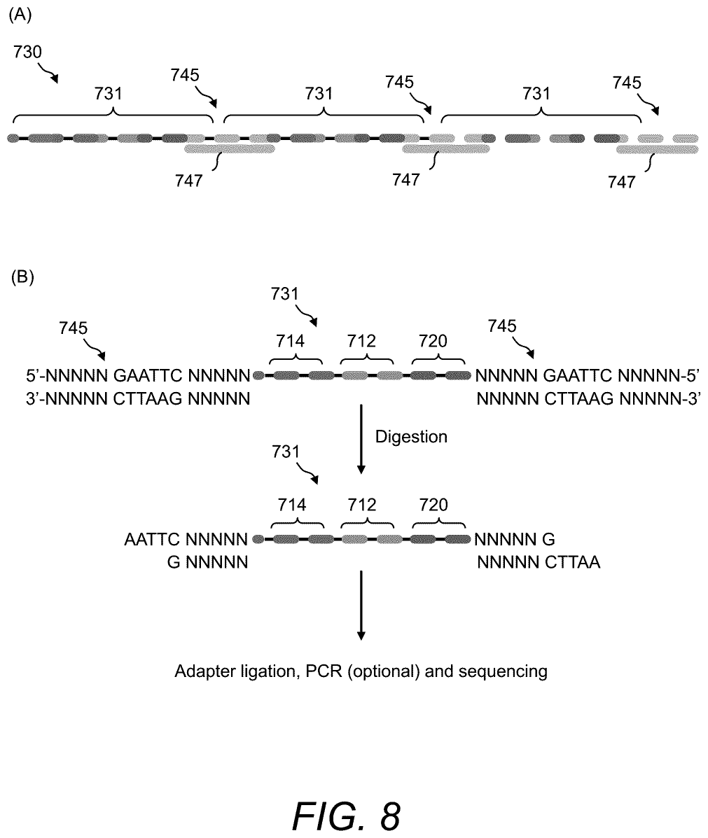

FIG. 8 is a schematic diagram of an example of a portion of nanoball 730 of FIG. 7 that includes restriction sites that may be used to separate repeated copies of the probe in the nanoball. Referring to panel “A”, in this example, nanoball 730 includes three probe copies 731 that may be separated by cleavage at a restriction endonuclease site 745. A restriction site (RS) complementary sequence 747 may be hybridized to restriction sites 745 to provide a double-stranded region for cleavage.

Referring to panel “B”, restriction sites consist of a recognition sequence and flanking bases to ensure that strands remain hybridized after cleavage. Flanking sequences (NNNNNN) may be of length ranging from about 5 to about 50 bases and can be designed to minimize interactions with other probe components and tune the melting temperature (Tm). In this example, the flanking sequences include five bases (N). The RS sequences can be used as an SBS primer such that sequencing begins with the code or may include a spacer region that is read prior to the code.

Digestion of nanoball 730 hybridized to RS complementary sequences 747 yields many code-containing DNA fragments with termini that contain single-stranded DNA overhangs or “sticky ends”. The digestion products may be further processed for sequencing. For example, adapters may be ligated to the sticky ends resulting from the restriction digestion.

Alternatively, the ends may be blunt ended (i.e., the single-stranded overhangs removed) and prepared for ligation to adapters. Blunt ended fragments may then be processed via typical sequencing sample preparation protocols such as A-tailing and adapter ligation.

An additional embodiment includes using a primer and polymerase to create RCA products where the entire concatemer is double stranded. This structure can then be processed via the restriction endonuclease procedure described above.

Another embodiment includes employing hyperbranched RCA to create many double stranded, code-containing sequences that can be processed via the restriction endonuclease procedure described above.

In certain embodiments, the restriction endonuclease may be a member of the cas family of proteins or a derivative thereof. These proteins recognize longer sequences of DNA, making them more specific.

In an additional embodiment, circularized probes may be prepared for sequencing without RCA.

In certain embodiments, the nanoballs of the invention may be compacted prior to sequencing. Rolling circle amplification produces linear concatemers of single-stranded DNA. When the substrate for RCA is a circularized probe, these concatemers may contain 100s-1000s of copies of a code. When preparing RCA products for sequencing, it is useful to compact them. The compacting may produce spherical structures. The compacted structures can increase localization of signal.

Compaction of RCA products into spherical nanoballs can be accomplished by a variety of techniques. In one embodiment, cationic additives that condense high molecular weight DNA (e.g., spermidine, Mg ions, cationic polymers) may be used. The compactness of a spherical nanoball may be tuned by controlling the concentration of the cationic reagent used. The concentration of the cationic reagent used may be selected to avoid aggregation of multiple nanoballs.

In one embodiment, multivalent oligonucleotide sequences that crosslink sites on RCA products may be used to compact RCA products into spherical nanoballs. The RCA binding sites may be separated by a nucleic acid or polymeric linker to control the degree of compaction. The compactness of the spherical nanoball may, for example, be tuned by controlling the degree of crosslinking in the RCA product.

In one embodiment, incorporation of modified nucleotides followed by crosslinking may be used to compact RCA products into spherical nanoballs. Examples of modified nucleotides that may be used include biotinylated nucleotides that bind to streptavidin proteins and nucleotides that covalently react with multifunctional linkers (e.g., amino nucleotides and NHS-terminated linkers). The compactness of the spherical nanoball may, for example, be tuned by controlling the degree of crosslinking in the RCA product.

In certain embodiments, the assays of the invention make use of nanopore sequencing. A nanoball or a circular modified probe may be sequenced using nanopore sequencing. Various nanopore sequencing sample preparation techniques are known in the art. Amplification is optional. Various components required for other sequencing techniques, such as sequencing primers, may be omitted from the probe. Purification can be accomplished using, for example, SPRI beads or BluePippen. Oxford Nanopore Technologies, Inc. (Oxford, UK) provides kits for sample preparation. Examples include Ligation Sequencing Kit, Native Barcoding Kit 96, and Rapid Barcoding Kit.

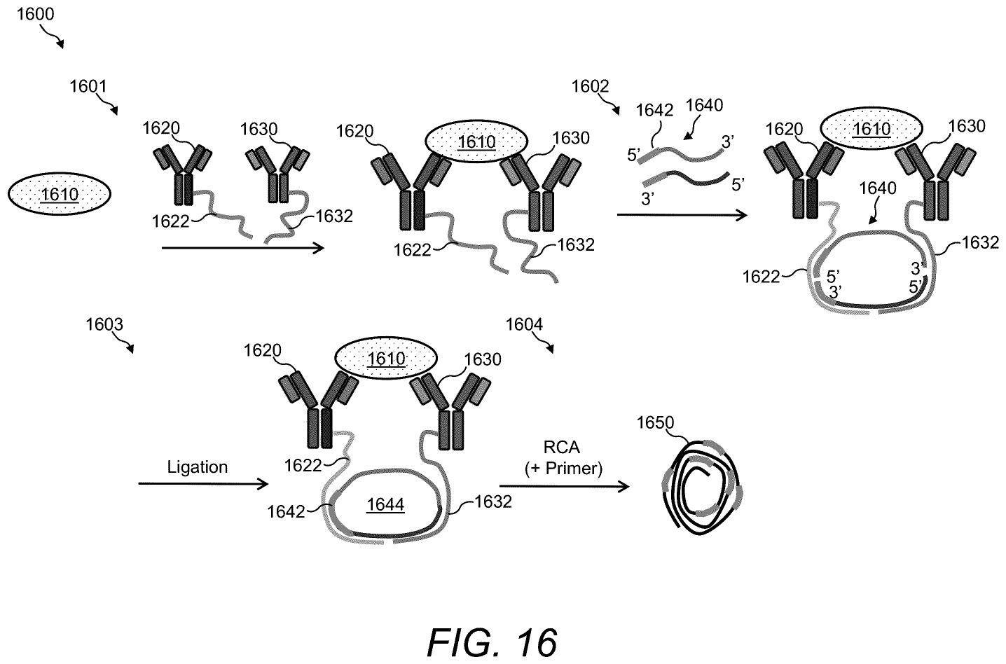

In certain embodiments, it may be useful to further amplify RCA products prior to sequencing. For example, in applications that use cell-free DNA (cfDNA) as the input where the analyte number may be low, it may be useful to amplify the RCA product prior to sequencing. In one embodiment, a circle-to-circle amplification approach may be used to produce multiple RCA products from one initial RCA product by monomerization of the concatemer (i.e., cleavage to unit length fragments), recircularization of the unit length fragments (i.e., monomers) and amplification of the newly generated circles in a second RCA reaction to produce multiple RCA product copies for further processing or sequencing. The restriction enzyme approach described with reference to FIG. 16 may be used to digest the initial RCA product to unit length (i.e., monomers). In some cases, an end-to-end joining oligonucleotide plus an end-to-end ligation reaction may be used to circularize the unit size fragments.

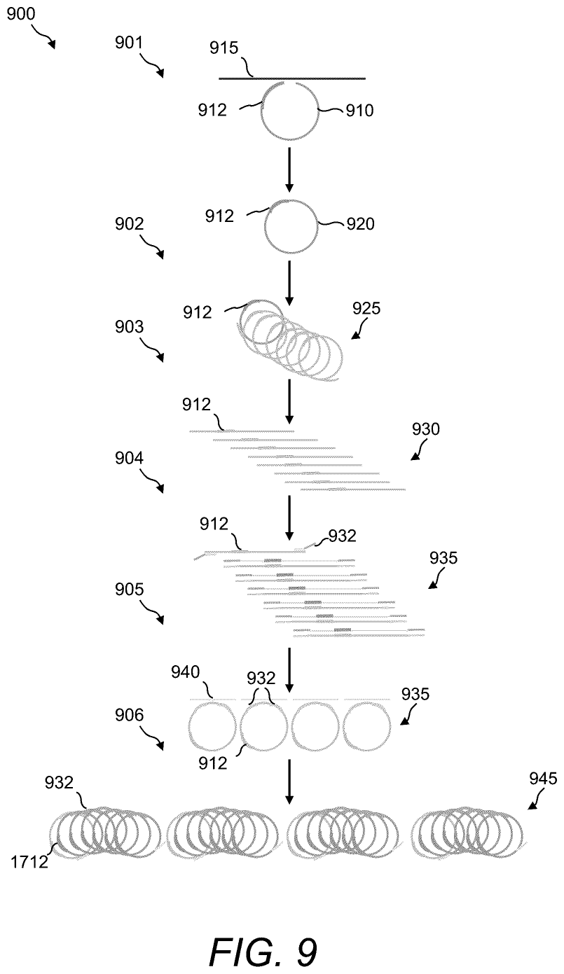

FIG. 9 is a schematic diagram of an example of a process 900 for circularizing and amplifying unit length nanoball fragments to produce multiple RCA nanoball products. Workflow 900 may include, but is not limited to, the following steps.

In a step 901, a probe is hybridized to a target and circularized to yield a circular modified probe. For example, a probe 910 that includes a code 912 and a restriction site (not shown) is hybridized to target 915. A ligation reaction is then performed to circularize probe 910 to produce a circular modified probe 920.

In a step 902, the circular modified probe 920 is amplified in an RCA reaction to generate a nanoball product 925. During amplification, the restriction site is amplified into the nanoball and provides multiple sites at which to cut nanoball 925 into fragments.

In a step 903, the nanoball product is cleaved to produce multiple unit sized fragments each comprising the code. For example, nanoball 925 is cleaved at the restriction sites to produce multiple unit size fragments 930 each comprising code 912. The cleavage reaction may, for example, be performed as describe with reference to FIG. 8.

In a step 904, the unit size fragments are amplified in a PCR reaction to generate multiple double-stranded fragments. For example, indexed amplification primers 932 are hybridized to unit size fragments 930 and a PCR reaction is performed to produce multiple unit size fragments 935 that include code 912 and the indexed amplification primer 932.

In a step 905, the amplified unit size fragments are circularized to generate circular unit size fragments. For example, an end-to-end joining oligonucleotide 940 that is complementary to sequences in amplification primer 932 is hybridized to unit size fragment 930 and an end-to-end ligation reaction is performed to generate circular unit size fragments 935 comprising the code.

In a step 906, the circular unit size fragments are amplified in a second RCA reaction to produce multiple nanoball copies for further processing or sequencing. For example, circular unit size fragments 935 are amplified in an RCA reaction to produce multiple nanoballs 945 each comprising code 912 and indexed amplification primers 932.

In an embodiment of process 900 of FIG. 9, the PCR amplification step 904 may be omitted and the unit size fragments comprising the code may be re-circularized for subsequent amplification in a second RCA reaction.

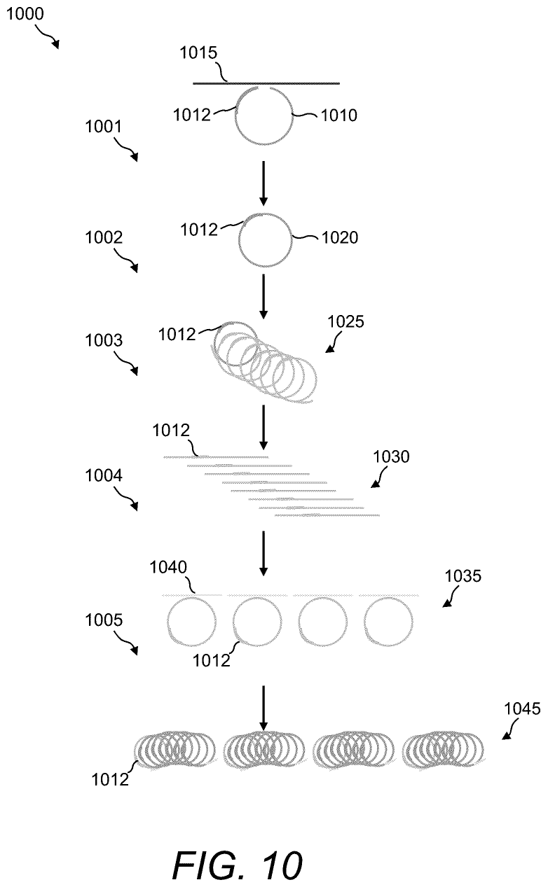

FIG. 10 is a schematic diagram of an example of an alternative process 1000 for circularizing and amplifying unit length nanoball fragments to produce multiple RCA nanoball products. Workflow 1000 may include, but is not limited to, the following steps.

In a step 1001, a probe is hybridized to a target and circularized to yield a circular modified probe. For example, a probe 1010 that includes target recognition sequences (not shown), a code 1012 and a restriction site (not shown) is hybridized to a target 1715. A ligation reaction is then performed to circularize probe 1010 to produce a circular modified probe 1020.

In a step 1002, the circular modified probe 1020 is amplified in an RCA reaction to generate a nanoball product 1025. During amplification, the restriction site is amplified into the nanoball and provides multiple sites at which to cut nanoball 1025 into fragments.

In a step 1003, the nanoball product is cleaved to produce multiple unit sized fragments each comprising the code. For example, nanoball 1025 is cleaved at the restriction sites to produce multiple unit size fragments 1030 each comprising code 1012. The cleavage reaction may, for example, be performed as describe with reference to FIG. 8.

In a step 1004, the unit size fragments are circularized to generate circular unit size fragments. For example, a splint oligonucleotide 1040 that is complementary to the target recognition sequences in unit size fragments 1030 is hybridized to the fragments and a ligation reaction is performed to generate circular unit size fragments 1035 comprising the code.

In a step 1005, the circular unit size fragments are amplified in a second RCA reaction to produce multiple nanoball copies for further processing or sequencing. For example, circular unit size fragments 1035 are amplified in an RCA reaction to produce multiple nanoballs 1045 each comprising code 1012.

Examples of sequencing techniques suitable for use with the assays disclosed herein include nanopore sequencing, next-generation sequencing, massively parallel sequencing, Sanger sequencing, sequencing by synthesis (SBS), pyrosequencing, sequencing by hybridization, single molecule real-time sequencing, SOLiD, and sequencing by ligation.

In some embodiments, a process for circularizing a probe may include a gap-fill ligation reaction that may be used to circularize the probe and capture an unknown region of the target that may then be sequenced along with the code.

### Assay Workflows

The assays provide a readout that can be measured alongside the readout of various molecular assays that may be performed in parallel, thereby enabling a multiomic platform for the analysis of different target analytes in a sample.

Examples of target analytes include, but are not limited to, proteins, nucleic acids (e.g., DNA and RNA), metabolites, glycosylation, exosomes, viruses, bacteria, and cells (e.g., circulating tumor cells).

In various embodiments, an encoded assay may be performed for the analysis of a set of protein targets in a sample.

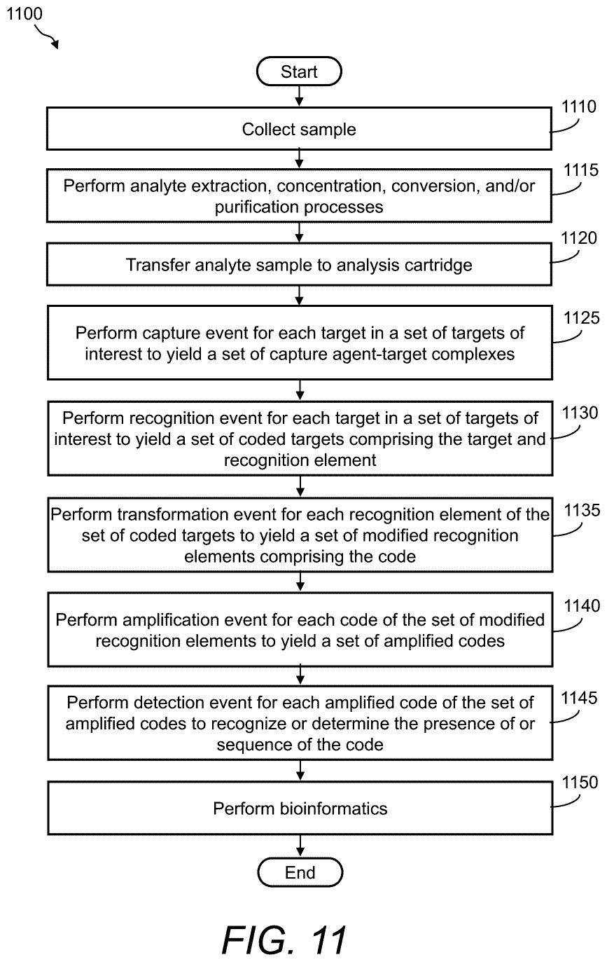

FIG. 11 is a flow diagram of an example of a targeted analyte assay workflow 1100. Assay workflow 1100 may include, but is not limited to, the following steps.

At a step 1110, a sample is collected. For example, a blood or saliva sample may be collected. In one example, a whole blood sample may be collected and processed to separate the plasma fraction from the cellular components of whole blood.

At a step 1115, target analyte extraction, concentration, and/or purification processes are performed. In this example, the target is a protein.

At a step 1120, the processed protein sample is transferred into an analysis cartridge.

At a step 1125, a capture event for each target in a set of targets of interest is performed to yield a set of capture agent-target complexes. For example, in the capture event, each target in the set of targets is uniquely recognized and bound by a capture agent to form a capture agent-target complex.

In some embodiments, the capture agent may be a pair of antibodies that recognize and bind a target of interest. For example, a first antibody may be used to capture the target and a second antibody may be used to bind a recognition element comprising a code with the captured target. In this case, the capture agent-target complex includes the first and second antibodies and the target of interest.

In one embodiment, the capture agent may be an aptamer (e.g., an oligonucleotide or peptide sequence) that is specific for a target of interest. Binding of the aptamer with the target forms an aptamer-target complex. In one embodiment, the aptamer or a sequence related to the aptamer (e.g., an extension of the aptamer) may be read as a code.

At a step 1130, a recognition event for each capture agent-target complex of the set of targets is performed. For example, in the recognition event, each capture agent-target complex is uniquely recognized by a recognition element associated with a code (and optionally other elements). In one embodiment, the recognition event for the set of targets uses a panel of coded padlock probes. The recognition event yields a set of coded targets comprising the capture agent-target complex and the recognition element.

At a step 1135, a transformation event for each recognition element of the set of coded targets is performed. For example, in the transformation event, a ligation or a gap-fill ligation may be used to produce the modified recognition element, i.e., a version of the recognition element that is ligated or gap-filled. In one example, transformation of a padlock probe in a ligation or gap-fill ligation reaction may be used to generate a circular modified probe. In some cases, an exonuclease cleanup step may be used following the transformation event to digest any remaining single stranded nucleic acid, such as unreacted coded padlock probes. The transformation event yields a set of modified recognition elements comprising the codes.

At a step 1140, an amplification event for each code of the set of modified recognition elements is performed. In one example, the amplification event may be a rolling circle amplification (RCA) reaction to generate a set of target-specific nanoballs. The amplification event yields a set of amplified codes (among other elements).

At a step 1145, a decoding event for each amplified code of the set of amplified codes is performed to recognize or determine the sequence of the code. In one example, the code may be determined by sequencing the code (and optionally other elements). The decoding event detects the code as a surrogate for detection of the target analyte.

At a step 1150, using the code information (and optionally other elements) from step 445, bioinformatics is performed.

In some embodiments, the amplification event (step 1135) and the decoding event (step 1140) may be combined in a single step.

### Surface-Bound Assays

In some embodiments, an encoded assay for detection of a target protein may be a surface-bound assay, wherein a capture agent is immobilized on a surface. The surface may, for example, be a well of a microtiter plate.

A surface-bound assay may, for example, be a modification of a standard immuno-detection assay, such as a sandwich ELISA assay. For example, a first antibody is used to capture a target of interest, and a second antibody and a linking agent are used to associate a recognition element with the target.

FIG. 12 is a schematic diagram illustrating an example of a modified ELISA process 1200 of the invention.

In a step 1201, a capture antibody 1210 immobilized on a surface 1212 is used to bind and immobilize a target protein 1214 of interest. Capture antibody 1210 may, for example, be immobilized on surface 1212 via covalent bond. A second antibody 1216 is then bound to the captured target 1214 to form antibody-target complex for detection of the target of interest. Second antibody 1216 may include a biotin moiety 1218.

In a step 1202, an intermediary agent 1220 is then used to link the antibody-target complex to an oligonucleotide tag. In this example, intermediary agent 1220 is streptavidin. A biotinylated oligonucleotide tag 1230 that includes complementary sequence to an encoded probe is linked to the antibody-target complex by the formation of a biotin-streptavidin linkage.

In a step 1203, an encoded probe 1232 is hybridized to oligonucleotide tag 1230. In one example, encoded probe 1232 is a padlock probe.

In a step 1204, a ligation reaction is performed to circularize encoded probe 1232 to yield a circular modified encoded probe and an RCA reaction is performed to generate a nanoball product 1240.

In another example (not shown), the capture antibody (i.e., capture antibody 1210) may be immobilized on the surface via a biotin/streptavidin linkage or via another noncovalent method. For example, streptavidin may be covalently linked to the surface and the capture antibody may be a biotinylated capture antibody.

In one embodiment, a surface-based protein assay workflow may include the following steps:
(i) capturing and immobilizing a target protein on a surface bound capture agent (i.e., a first antibody);
(ii) binding a biotinylated second antibody to the target to form a capture agent-target complex;
(iii) binding a biotinylated oligonucleotide tag to the second antibody via a biotin-streptavidin linkage to form a capture agent-target-oligonucleotide tag complex;
(iv) hybridizing an encoded probe to the oligonucleotide tag;
(v) performing a ligation reaction to circularize the encode probe to yield a modified circular probe;
(vi) performing a rolling circle amplification (RCA) reaction to amplify the code and generate a nanoball product; and
(vii) determining (identifying) the presence of or the sequence of the code.

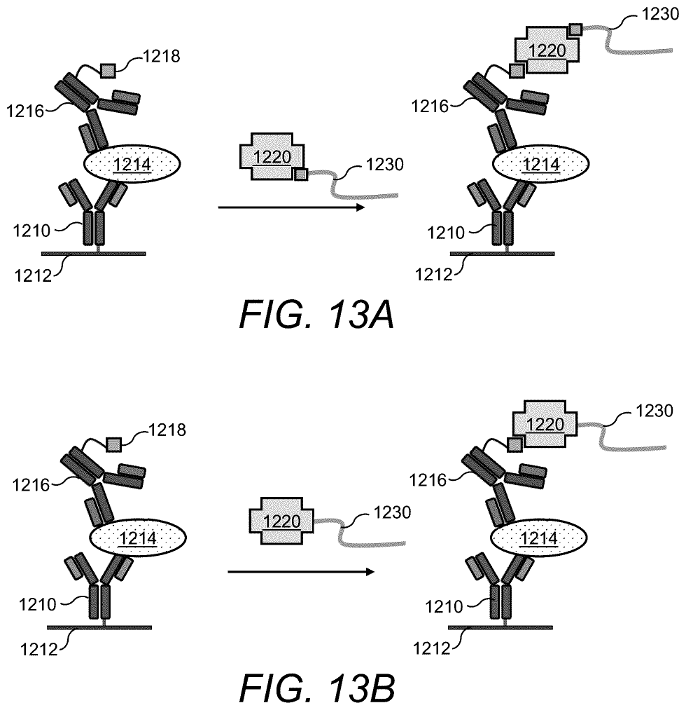

FIG. 13A and FIG. 13B are schematic diagrams illustrating two examples of a modified sandwich ELISA assay that may be used for the detection of target biomolecules in a surface-bound assay. FIG. 13A shows target protein 1214 immobilized by capture antibody 1210 on surface 1212 and bound by a biotinylated second antibody 1216 to form an antibody-target complex. In this example, the intermediary agent 1220 (streptavidin) is pre-conjugated to oligonucleotide tag 1230 via a biotin-streptavidin linkage. The pre-conjugated streptavidin-oligonucleotide tag 1230 is then bound to the antibody-target complex to form an oligonucleotide-streptavidin-antibody-target complex.

In an alternative embodiment (not shown), free streptavidin (SA) may bind to the antibody-target complex and then a biotinylated oligonucleotide tag that is complementary to an encoded probe is bound to the streptavidin-antibody-target complex.

FIG. 13B shows an alternative approach that uses an oligonucleotide tag 1230 pre-conjugated to intermediary agent 1220 (e.g., streptavidin) via a covalent bond to form an oligonucleotide-streptavidin-antibody-target complex.

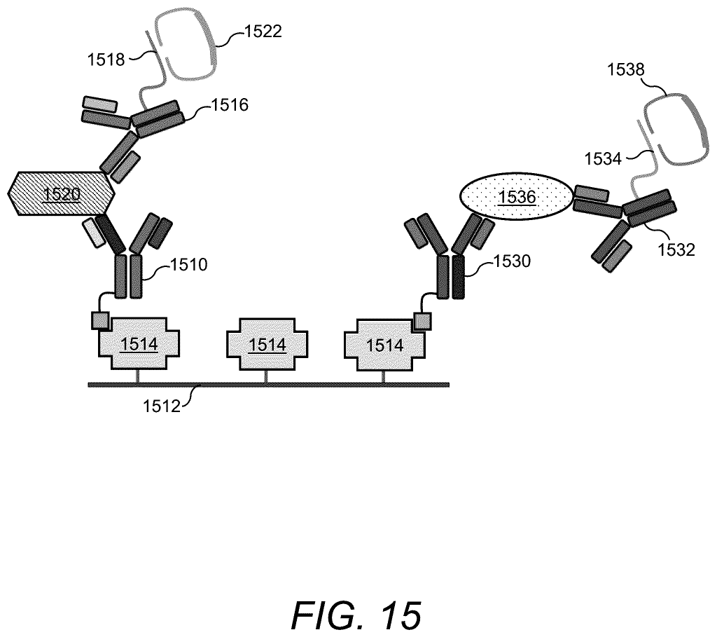

In another embodiment, a surface may be coated with streptavidin to bind a target specific capture agent and a second target specific binding agent conjugated to an oligonucleotide tag is used to link the capture agent-target complex (e.g., an antigen-antibody complex) to an encoded probe readout. Conjugation of an oligonucleotide tag to a second target specific binding agent, e.g., a monoclonal antibody, provides for multiplexed detection of multiple targets in an assay. More details of multiplexed detection is described hereinbelow with reference to FIG. 15.

FIG. 14 is a schematic diagram illustrating an example of a modified ELISA process 1400 that may be used for multiplexed detection of target biomolecules in a surface-bound assay.

At a step 1301, a surface 1310 that includes a streptavidin coating 1312 is provided and a capture antibody 1314 (e.g., a monoclonal antibody) that includes a biotin moiety 1316 is immobilized on surface 1312 via a streptavidin-biotin linkage.

At a step 1302, a sample is introduced and a target 1320 is bound by capture antibody 1314 and immobilized on surface 1310.

At a step 1303, a second antibody 1330 is introduced and bound to the captured target 1314 to form antibody-target complex for detection of the target of interest. Second antibody 1330 is conjugated to an oligonucleotide tag 1332 that includes a sequence that is complementary to sequence in an encoded probe (e.g., a padlock probe).

At a step 1304, an encoded probe 1340 is introduced and hybridized with oligonucleotide tag 1332.

At a step 1305, a ligation reaction is performed to circularize encoded probe 1340 and an RCA reaction is performed to amplify the code and generate a nanoball output product 1342.

FIG. 15 is a schematic diagram illustrating an example of multiplexing target detection in a surface-bound assay. In this example, two orthogonal monoclonal antibodies for each target protein are used: a first antibody is conjugated to biotin and a second antibody is conjugated to an oligonucleotide tag that is complementary to an encoded probe. For each target of interest, the encoded probe includes a unique target associated code and sequences complementary to the oligonucleotide tag. For example, a biotinylated antibody 1510 is anchored on a surface 1512 that includes a streptavidin coating 1514 via a streptavidin-biotin linkage. Biotinylated antibody 1510 and a second antibody 1516 that is conjugated to a first oligonucleotide tag 1518 are bound to a first target 1520. Oligonucleotide tag 1518 includes a sequence that is complementary to sequence in a first encoded probe 1522 (e.g., a padlock probe). A second biotinylated antibody 1530 is anchored on surface 1512 via a streptavidin-biotin linkage. Biotinylated antibody 1530 and a second antibody 1532 that is conjugated to a second oligonucleotide tag 1534 are bound to a second target 1536. Oligonucleotide tag 1534 includes a sequence that is complementary to sequence in a second encoded probe 1538 (e.g., a padlock probe).

### Solution-Based Assay

An assay for multiplexed detection of target biomolecules may be a solution-based assay, wherein the capture agent is free in solution.

In some embodiments, two different capture agents (e.g., antibodies) that are specific for different positions of a target of interest (e.g., a protein) and a split encoded probe may be used. For example, a first capture agent is conjugated to an oligonucleotide tag that includes sequences complementary to one fragment of the split encoded probe and the second capture agent is conjugated to a second oligonucleotide tag that is complementary to the other fragment of the split encoded probe. A proximity ligation reaction may then be used to ligate the two encoded probe fragments and generate a readable code sequence. In this embodiment, four binding events must occur to generate a readable code sequence, thereby limiting the number of random events that may generate an incorrect detection result.

In one example, a set of polyclonal antibodies may be used as capture agents, wherein one half of the polyclonal antibodies are conjugated to a first oligonucleotide tag and the other half of the polyclonal antibodies are conjugated to a second oligonucleotide tag.

In another example, two monoclonal antibodies with defined target binding sites may be used as capture agents, wherein a first monoclonal antibody is conjugated to a first oligonucleotide tag and the other monoclonal antibody is conjugated to a second oligonucleotide tag.

FIG. 16 is a schematic diagram illustrating an example of a solution-based assay 1600 that uses two different capture agents and a split encoded probe for the detection of a target of interest.

At a step 1601, a sample that may include a target 1610 is introduced and combined with two different capture antibodies: a first antibody 1620 that is conjugated to a first oligonucleotide tag 1622 and a second antibody 1630 that is conjugated to a second oligonucleotide tag 1632. Oligonucleotide tags 1622 and 1632 include sequences that are complementary to two fragments in a split probe 1640. Each fragment of split probe 1640 includes a portion of a code 1642.

At a step 1602, oligonucleotide tags 1622 and 1632 are hybridized to the two fragments of split encoded probe 1640.

At a step 1603, a proximity ligation reaction is performed to ligate the two fragments of encoded probe 1640 to form a circularized encoded probe 1644 and generate a readable code 1642 sequence.

At a step 1604, an RCA reaction is performed to amplify code 1642 and generate a nanoball output product.

In another embodiment of assay 1600, one antibody capture agent may be bound to a substrate surface and the second antibody capture agent may be provided in a reaction solution. Binding of one antibody capture agent to the substrate surface may be used to facilitate subsequent clean up and/or isolation steps in the assay.

In another embodiment of assay 1600, an encoded probe may be split in other ways than through the code sequence. For example, the code sequence of a split encoded probe may be intact on either half of the encoded probe.

In some embodiments, two orthogonal capture agents (e.g., antibodies) that recognize and bind to different regions of a target of interest may be used to associate a code with a target of interest. For example, a split encoded probe may be attached via a linker to two orthogonal capture agents (e.g., antibodies), wherein one capture agent is linked to one half of the split encoded probe and the other capture agent is linked to the other half of the split encoded probe. The linkage between an encoded probe fragment and a capture agent may be cleavable or non-cleavable. A pair of bridging oligonucleotides may then be used to facilitate a ligation reaction to generate a readable code sequence. In this approach, four separate events are required to generate a readable code, i.e., two antibody binding events and two hybridization/ligation events. Because four separate events are required to generate a readable code, the number of random events that may occur during generation of a readable code are reduced.

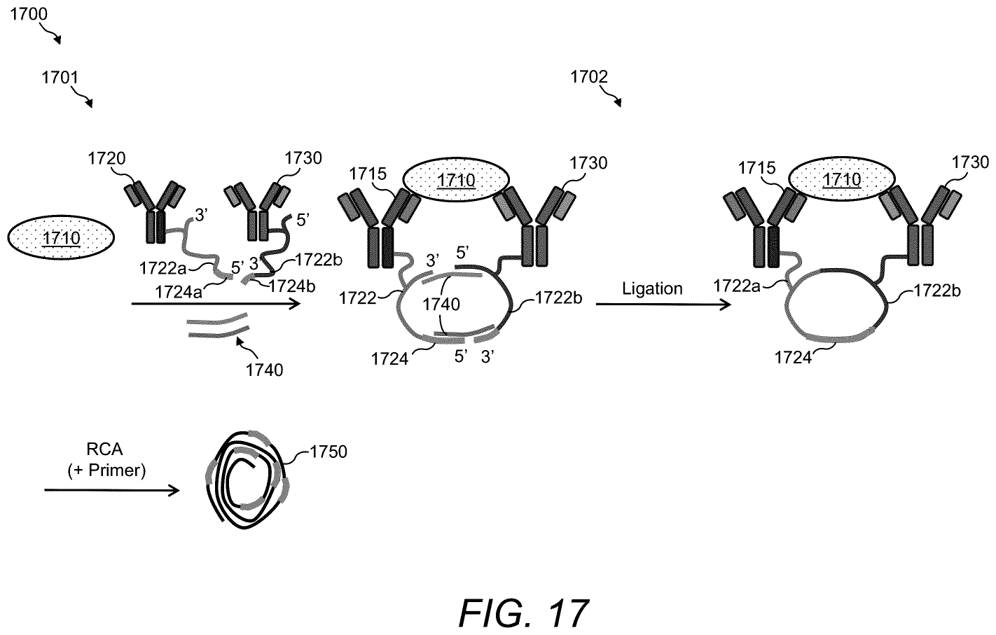

FIG. 17 is a schematic diagram illustrating an example of a process 1700 for detecting a target of interest using a split encoded probe integrated with the capture agents and a pair of bridging oligonucleotides to facilitate a ligation reaction to generate a readable code.

At a step 1701, a sample that may include a target 1710 is introduced and combined with two different capture antibodies and a pair of bridging oligonucleotides. For example, a first antibody 1720 that is conjugated to a first portion of a split probe 1722 (e.g., 1722a) and a second antibody 1730 that is conjugated to a second portion of split probe 1722 (e.g., 1722b) are combined in a binding reaction for binding to target 1710. Each portion of split probe 1722 (i.e., split probe 1722a and 1722b) includes a portion of a code 1724 (e.g., 1724a and 1724b, respectively). A pair of bridging oligonucleotides 1740 that include sequences complementary to split probe 1722 are introduced and hybridized to each portion of split probe 1722.

At a step 1702, a proximity ligation reaction is performed to ligate two fragments of split probe 1722 (i.e., split probe 1722a and 1722b) to form a circularized split probe 1722 and generate a readable code 1724 sequence.

At a step 1703, an RCA reaction is performed to amplify code 1724 and generate a nanoball output product 1750.

In another example, a single bridging oligonucleotide may be used for one ligation event and CircLigase may be used for the other ligation event.

In some embodiments, the linker used to couple the split encoded probe to each capture agent (e.g., antibodies) may be a cleavable linker that may be cleaved to separate the circularized encoded probe from the capture agents prior to the RCA reaction.

In another embodiment, a split encoded probe may be attached via a linker to two orthogonal capture agents (e.g., antibodies), wherein one capture agent is linked to one half of the split encoded probe and the other capture agent is linked to the other half of the split encoded probe, and a single bridging oligonucleotide may be used to facilitate a ligation reaction to generate a readable code sequence.

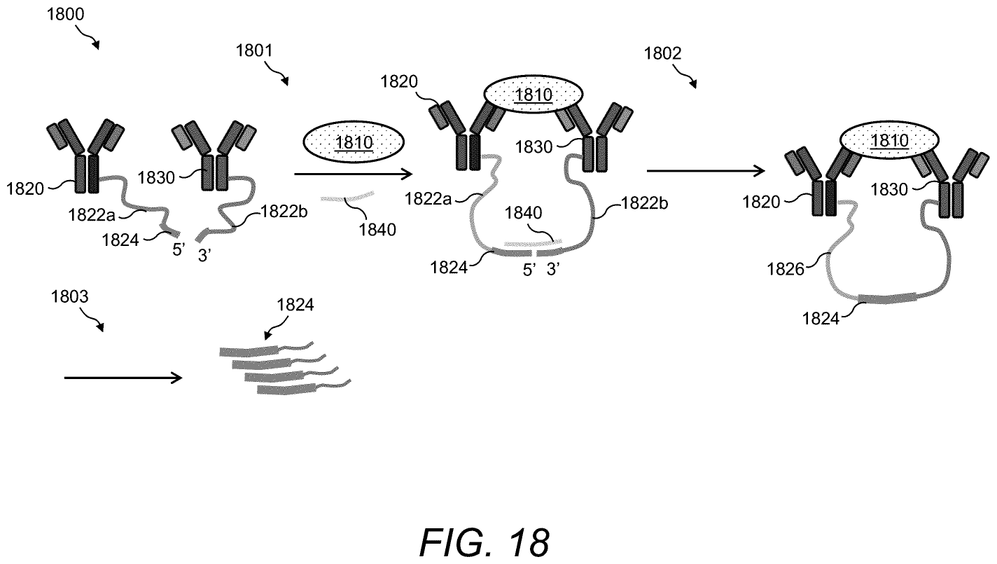

FIG. 18 is a schematic diagram illustrating an example of a process 1800 for detecting a target of interest using a split encoded probe integrated with the capture agents and a single bridging oligonucleotide to facilitate a ligation reaction to generate a readable code.

At a step 1801, a sample that may include a target 1810 is introduced and combined with two different capture antibodies and a bridging oligonucleotide. For example, a first antibody 1820 that is conjugated to a first portion of a split probe 1822 (e.g., 1822a) and a second antibody 1830 that is conjugated to a second portion of split probe 1822 (e.g., 1822b) are combined in a binding reaction for binding to target 1810. Each portion of split probe 1822 (i.e., split probe 1822a and 1822b) includes a portion of a code 1824 (e.g., 1824a and 1824b, respectively). A bridging oligonucleotide 1840 that include sequences complementary to split probe 1822 is introduced and hybridized to each portion of split probe 1822.

At a step 1802, a ligation reaction is performed to ligate the two fragments of split probe 1822 (i.e., split probe 1822a and 1822b) to form a ligated probe 1826 and generate a readable code 1824 sequence.

At a step 1803, a PCR reaction is performed to amplify code 1824 (among other sequences) and produce multiple linear amplified sequences that may be read to determine the code (e.g., read in a sequencing reaction).

A solution-based assay may be multiplexed for detection of two or more different targets in a sample. In one embodiment, two orthogonal capture agents (e.g., antibodies) for each target analyte are used: a first capture agent that is conjugated to a first oligonucleotide tag and a second capture agent that is conjugated to a second oligonucleotide tag, wherein each oligonucleotide tag includes a sequence that is complementary to a portion of a split probe. For each target of interest, the split probe includes a unique target associated code and sequences complementary to the oligonucleotide tags that are used to mediate the transformation of the split probe into an amplifiable probe comprising the code.

In one embodiment, two orthogonal capture agents (e.g., antibodies) for each target analyte are used: a first capture agent that is conjugated to a first portion of a split probe and a second capture agent that is conjugated to a second portion of the split probe. For each target of interest, the split probe includes a unique target associated code and sequences complementary to sequences that are used to mediate the transformation of the split probe into an amplifiable probe comprising the code.

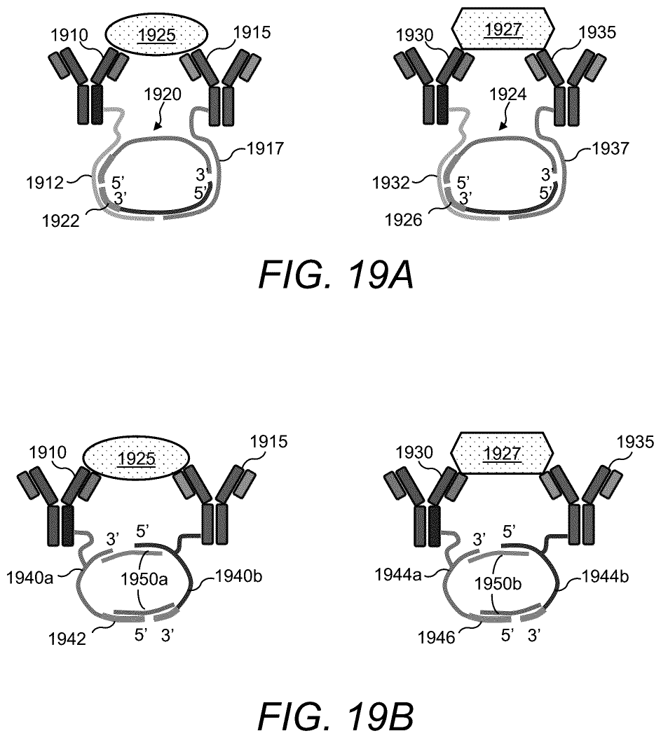

FIG. 19A and FIG. 19B are schematic diagrams illustrating examples of the assay components for multiplexing target detection in a solution-based assay. In FIG. 19A and 19B, the capture agent is an antibody, and the target analyte is a protein.

Referring now to FIG. 19A, in this example, two orthogonal capture antibodies, orthogonal oligonucleotide tags, and encoded split probes are used to detect two different targets 1925 and 1927 of interest as described above with reference to FIG. 16. For each target of interest (i.e., target 1925 and target 1927), the split probe includes a unique target associated code and sequences complementary to the oligonucleotide tags. For example, a first capture antibody 1910 is conjugated to a first oligonucleotide tag 1912 and a second capture antibody 1915 is conjugated to a second oligonucleotide tag 1917, wherein each oligonucleotide tag includes a sequence that is complementary to a portion of a split probe 1920. Split probe 1922 includes a unique target associated code 1922 and sequences complementary to the oligonucleotide tags 1912 and 1917. Similarly for detection of a second target 1927, a first capture antibody 1930 is conjugated to a first oligonucleotide tag 1932 and a second capture antibody 1935 is conjugated to a second oligonucleotide tag 1937, wherein each oligonucleotide tag includes a sequence that is complementary to a portion of a split probe 1924. Split probe 1924 includes a unique target associated code 1926 and sequences complementary to the oligonucleotide tags 1932 and 1937.

Referring now to FIG. 19B, in this example, two orthogonal capture antibodies, and encoded split probes are used to detect two different targets 1925 and 1927 of interest as described above with reference to FIG. 17. For example, a first antibody 1910 that is conjugated to a first portion of a split probe 1940 (e.g., 1940a) and a second antibody 1915 that is conjugated to a second portion of split probe 1940 (e.g., 1940b) are combined in a binding reaction for binding to target 1925. Each portion of split probe 1940 (i.e., split probe 1940a and 19402b) includes a portion of a code 1942 (e.g., 1942a and 1942b, respectively). A pair of bridging oligonucleotides 1950a that include sequences complementary to each portion of split probe 1940 are used to facilitate ligation of split probe 1940 to generate an amplifiable and readable code. Similarly for detection of a second target 1927, a first antibody 1930 is conjugated to a first portion of a split probe 1944 (e.g., 1944a) and a second antibody 1935 is conjugated to a second portion of split probe 1944 (e.g., 1944b) are combined in a binding reaction for binding to target 1925. Each portion of split probe 1944 (i.e., split probe 1944a and 19442b) includes a portion of a code 1946 (e.g., 1946a and 1946b, respectively). A pair of bridging oligonucleotides 1950b that include sequences complementary to each portion of split probe 1944 are used to facilitate ligation of split probe 1944 to generate an amplifiable and readable code.

### Soft Decoding

A soft decoding process may use decoding by hybridization (DBH).

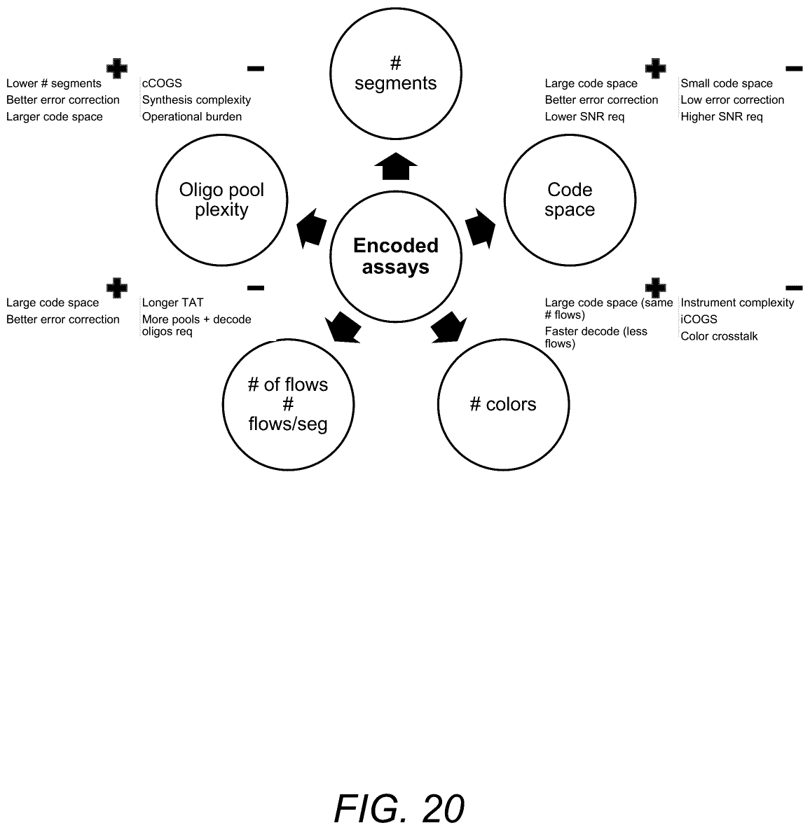

FIG. 20 is a schematic diagram illustrating some of the factors considered in the design of an encoded probe for decoding by hybridization.

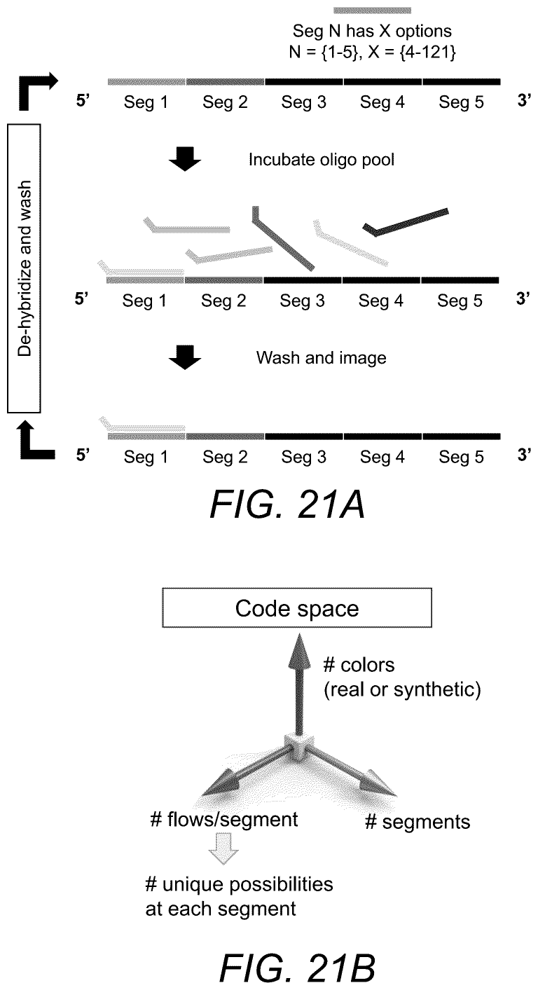

FIG. 21A is a schematic diagram illustrating an overview of process for decoding by hybridization. For example, a code may include 5 segments and decoding may use 1 flow/segment, 4 colors or oligonucleotides in the oligo pool/flow. The decoding by hybridization process may include repeated cycles of hybridizing a code sequence with a decoding oligonucleotide pool (decoding oligos) comprising fluorescently labeled oligos, washing the hybridization reaction to remove unbound decoding oligos, imaging the decoding reaction to determine the identity of the hybridized decoding oligo, and de-hybridizing the code sequence to initiate a subsequent decoding cycle.

FIG. 21B is a schematic diagram illustrating the code space in decoding by hybridization. For example, the code space may include the number of colors (real or synthetic), the number of flows per segment and the number of unique possibilities at each segment, and the number of segments in the code.

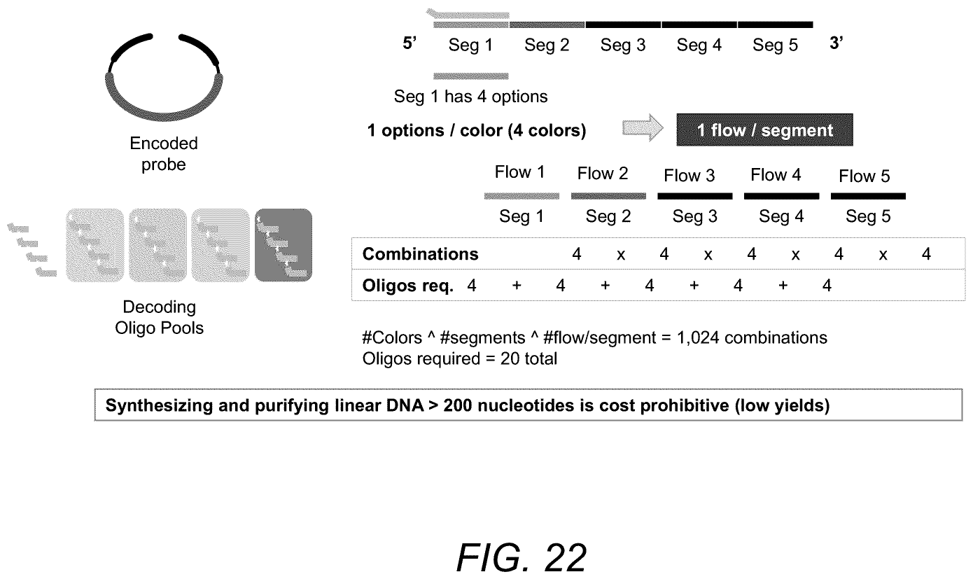

FIG. 22 is a schematic diagram of an example of a method for encoding symbols onto each segment of a code. In this example, the code comprises 5 segments (e.g., seg 1 through seg 5) which requires relatively few decoding oligos for decoding by hybridization. A code with 5 segments would require 5 decoding pools with 4 different labeled decoding oligos flowed for each segment decoded (i.e., 20 different decoding oligos are required).

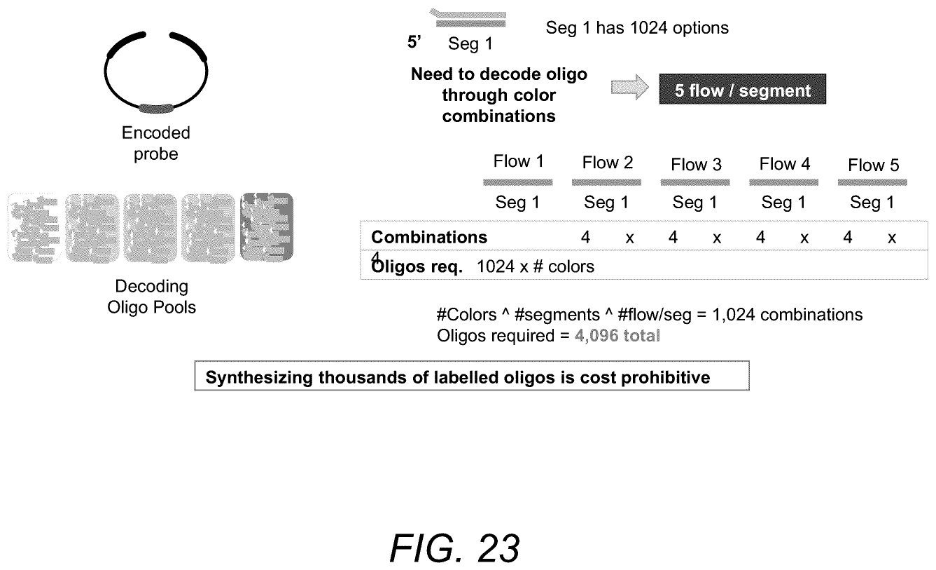

FIG. 23 is a schematic diagram of another example of a method for encoding symbols onto a code wherein the length of the code sequence comprises a single segment that requires a relatively large number of decoding oligos.

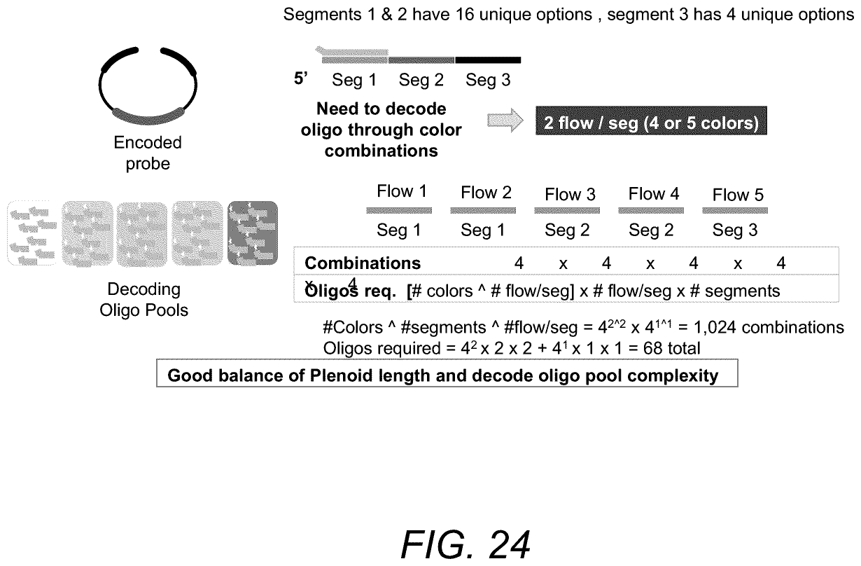

FIG. 24 is a schematic diagram of another example of a method for encoding symbols onto a code wherein the mix of segment number and flows/segment in the decoding process balances the length of a code and the complexity required in the decoding oligo pool.

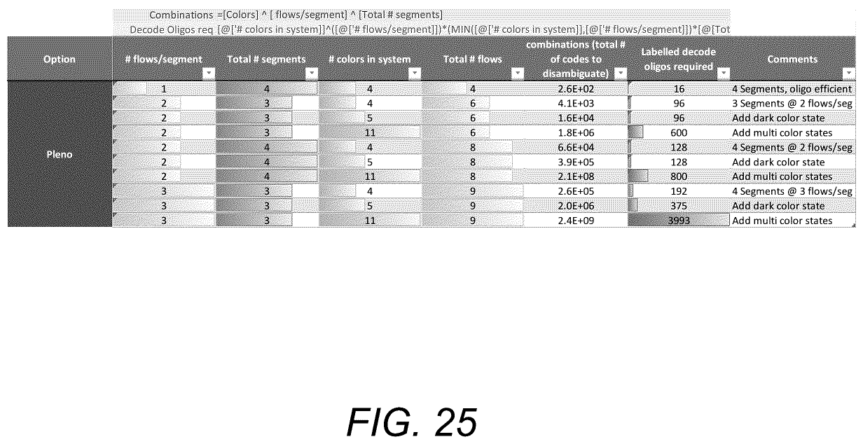

FIG. 25 is a screenshot of an example of the permutations (e.g., colors, flows/segment, total segments, and total flows) that may be used to achieve a relatively large combination space (codes pace) from which select a subset of codes.

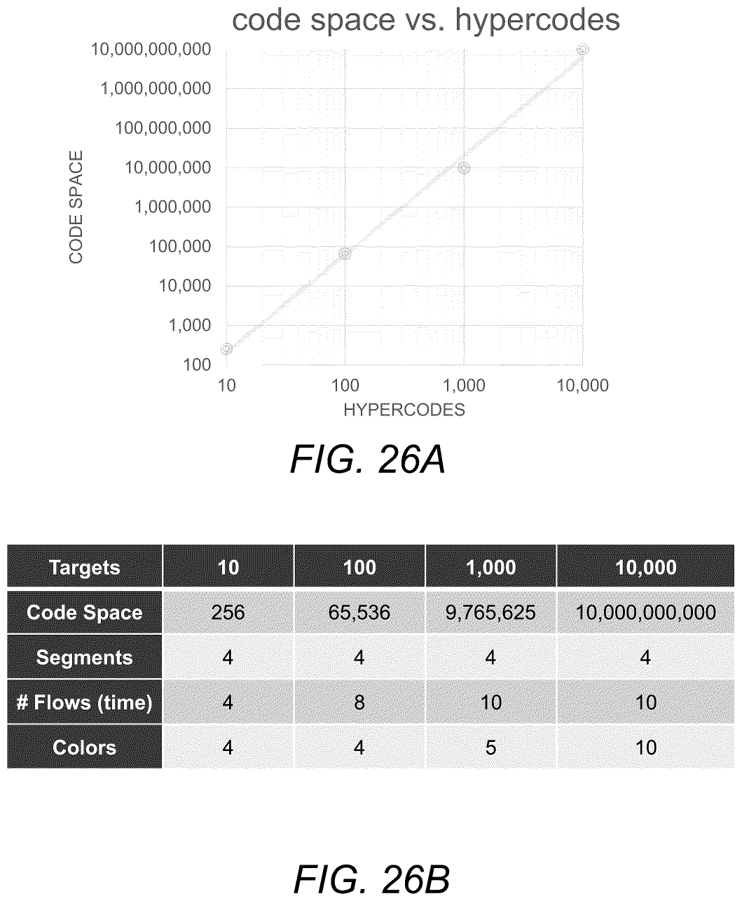

FIG. 26A and FIG. 26B are a plot showing the relationship of the number of codes in a code space, and a summary table of the number of segments, flows, and colors required for a given number of targets for detection, respectively.

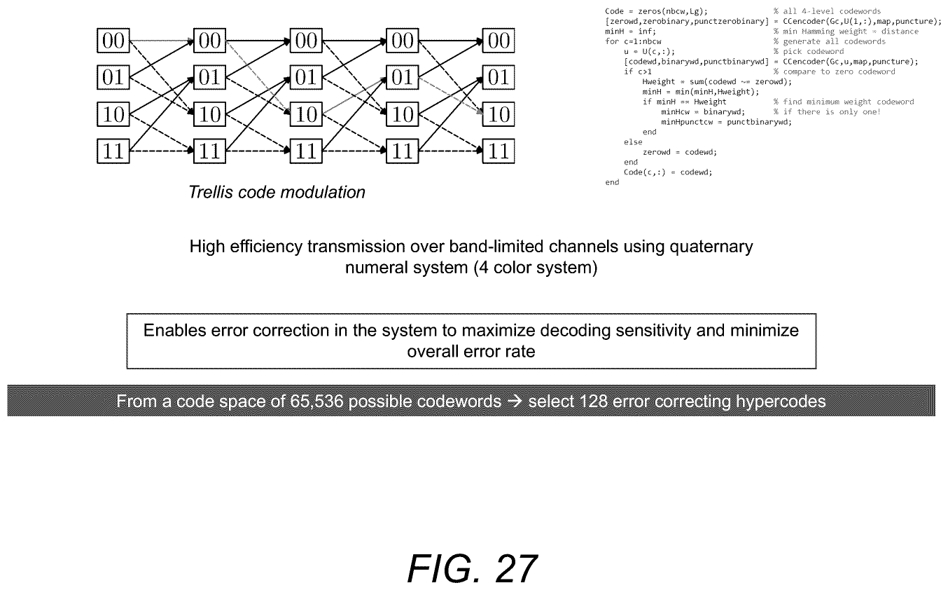

FIG. 27 is a schematic diagram of an example of a trellis code and a process of using the trellis code to select a set of codes with desired properties for an assay from a large code space. In this example, a 4-color system is used, which enables error correction in the system to maximize decoding sensitivity and minimize the overall error rate.

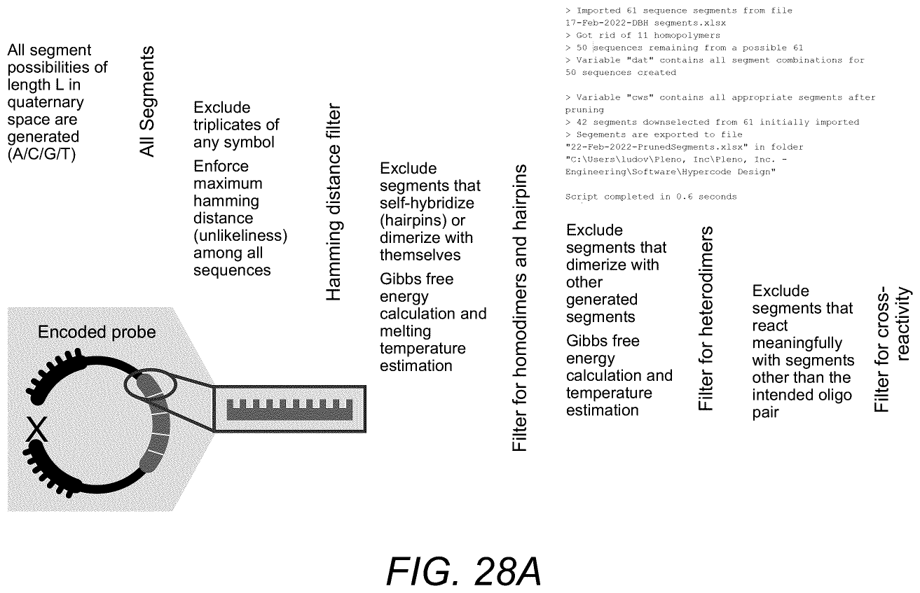

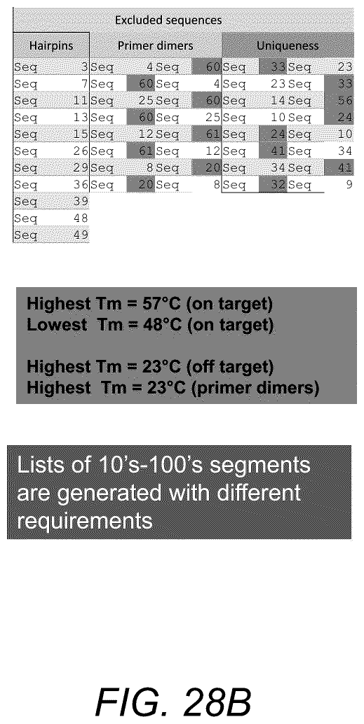

FIG. 28A and FIG. 28B are a representation of a strategy for designing oligo segments on a probe that will encode for the symbols that make up the trellis code (or other). The strategy may include translating the symbol from the code into the DNA backbone of the probe, either through 1 DNA base if sequencing, or decoding by (may be more than 1 base), or many bases if using decoding by hybridization (e.g., between 10-20 bases, though longer and shorter are possible).

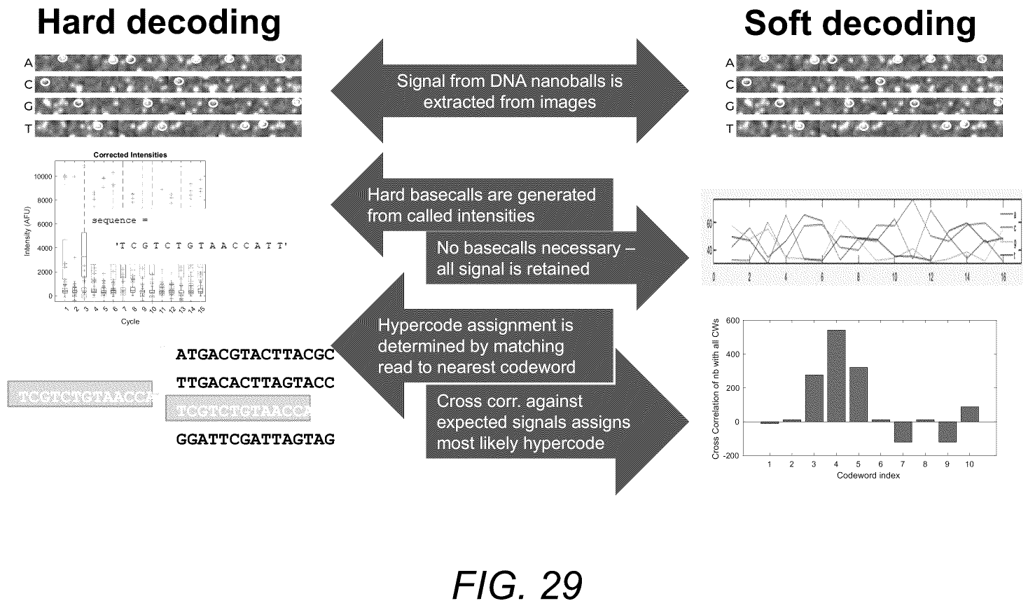

FIG. 29 is a representation of an overview of a decoding process comparing hard decoding vs. soft decoding.

FIG. 30 is a schematic diagram of an example of a soft decoding process that may be used in the assays of the invention.

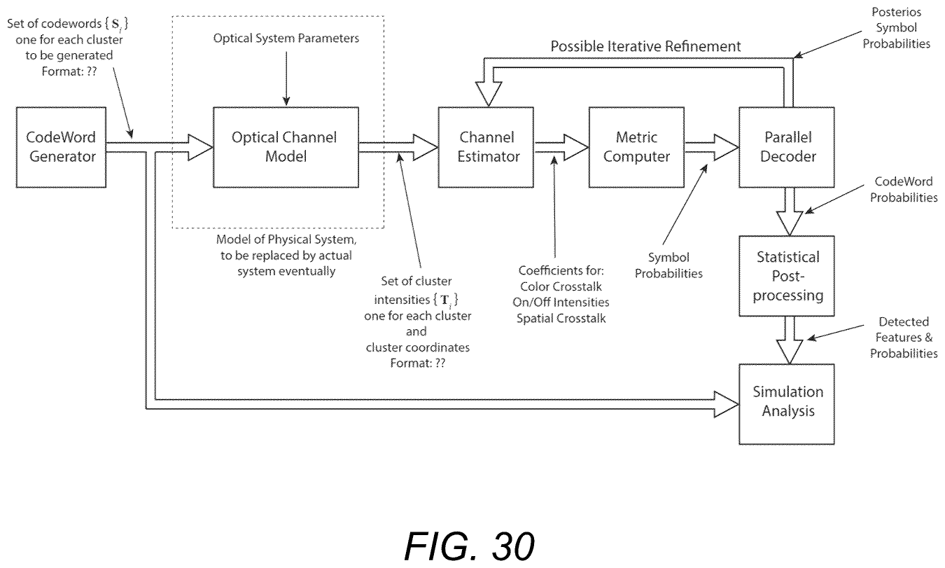

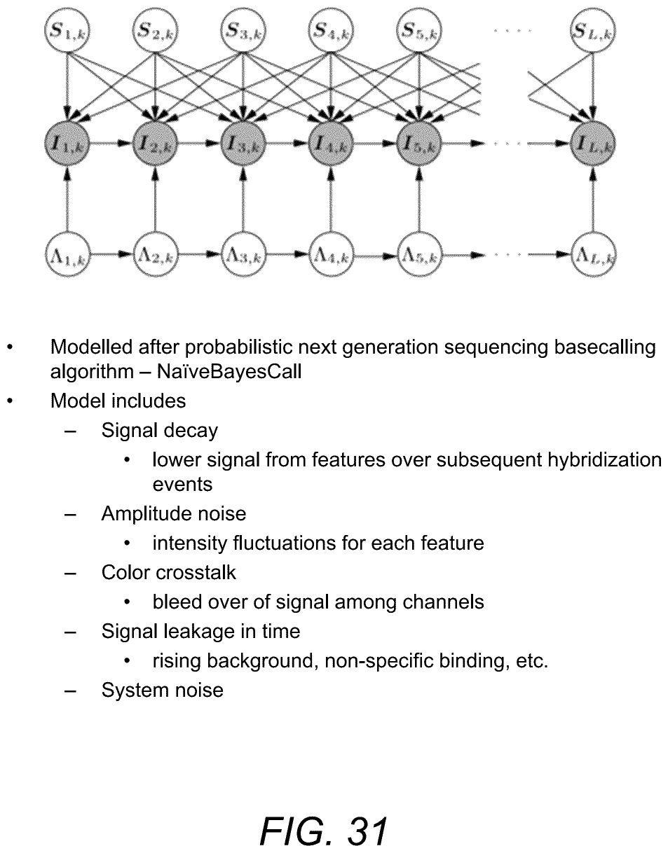

FIG. 31 is a summary of a channel model for a base calling algorithm that may be used in a soft decoding process. The model may include, for example, parameters for signal decay, amplitude noise, color crosstalk, signal leakage in time and system noise.

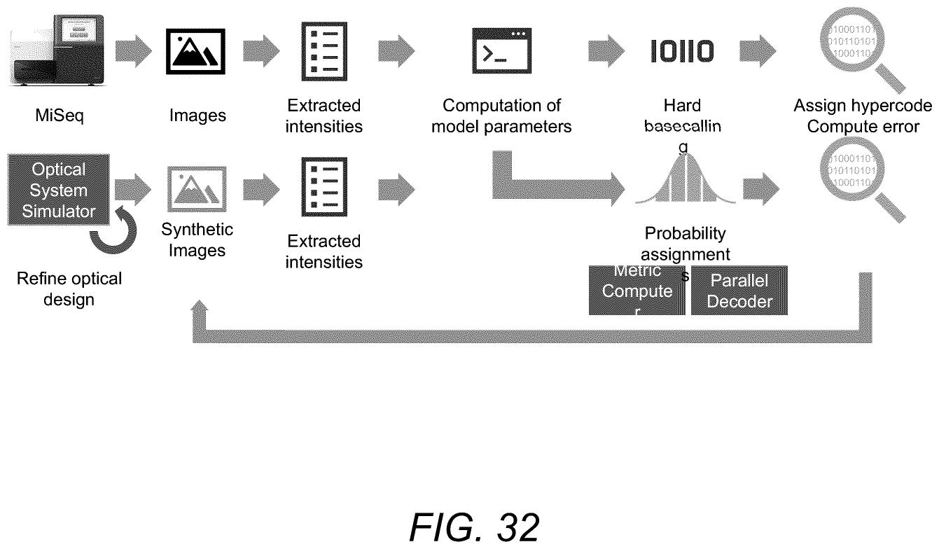

FIG. 32 is a schematic diagram illustrating an overview of an encoded assay analysis process.

In various embodiments of the invention, a method is provided for conducting an assay for polypeptide targets. The method includes: (a) binding each target in a sample that potentially includes a set of polypeptide targets to a capture agent to yield a set of capture agent-target complexes; (b) performing a recognition event on the capture agent-target complexes to yield capture agent-target complexes that include an encoded probe, the encoded probe having a code from a set of codes, each code having at least one segment encoding one or more symbols that correspond to a sequence of one or more nucleotides; (c) performing a molecular transformation event to produce modified encoded probes in the presence of the target and unmodified encoded probes in the absence of the target, in which the modified probes can be amplified and the unmodified probes cannot be amplified in an amplification event; and (d) performing the amplification event and detecting the targets by decoding the codes that are amplified.

In some instances, the codes that are amplified are decoded using a soft decision decoding method. For example, decoding the codes that are amplified may include recording signal produced in response to interrogation of each segment of the codes and, upon completion of the interrogation, determining a probably of the presence of each of the codes by applying a soft-decision probabilistic decoding algorithm to the recorded signal. The signal produced may include, but is not limited to, signal from one or a combination of nanopore sequencing, next-generation sequencing, massively parallel sequencing, Sanger sequencing, sequencing by synthesis (SBS), pyrosequencing, sequencing by hybridization, decoding by hybridization, single molecule real-time sequencing, SOLiD, and sequencing by ligation.

For the codes, each segment may comprise one symbol corresponding to one nucleotide. In one instance, each of the codes includes up to 50 segments for a length of each code up to 50 nucleotides. In this instance, decoding the codes that are amplified may include using sequencing by synthesis (SBS).

In other instances, each segment includes one symbol corresponding to more than one nucleotide.

In various embodiments, each code may include two or more segments, three or more segments, four or more segments, or five to sixteen segments.

In one instance, interrogation of the segments includes decoding by hybridization. At least one of the segments may be interrogated more than one time by hybridization with one or more hybridization probes each having at least one label to produce the signal. In some cases, at least four different labels may be utilized in the decoding by hybridization. The label may be an optical label or a fluorescent label.

In one example, each code includes at least four segments and at least sixteen symbols.

In the methods of the invention, the unique number of possibilities at each of the segments includes up to the number of different labels raised to the power of the number of the hybridizations per segment.

In one embodiment, at least one probe has two or more of the labels to create a pseudo label and generate a larger number of the symbols.

In the methods described herein, the set of targets may include tens of target analytes, hundreds of target analytes, thousands of target analytes, or tens of thousands of target analytes.

### EXAMPLES

A proof-of-concept (POC) experiment was performed to demonstrate the feasibility of using a modified sandwich ELISA assay for the detection of a protein target of interest. In this POC assay, a commercially available ELISA kit for the detection of the epithelial CA-125 protein and the Cyfra21-1 fragment of cytokeratin 19 (an epithelial marker protein) were used. The modified POC protein assay included the following steps:
(i) capture and immobilize target protein (antigen) to surface bound capture antibody;
(ii) bind biotinylated detection antibody to form an antigen-antibody complex;
(iii) bind biotinylated target-specific oligonucleotide to detection antibody via streptavidin bridge to form an antigen-antibody-target specific oligonucleotide complex;
(iv) hybridize encoded oligonucleotide probe to target-specific oligonucleotide;
(v) perform ligation reaction and rolling circle amplification (RCA) to generate nanoballs;
(vi) quantify RCA nanoball product by quantitative PCR (qPCR); and
(vii) generate digital readout of the code associated with the captured target protein of interest.

An example of an ELISA plate protein assay protocol is shown below.

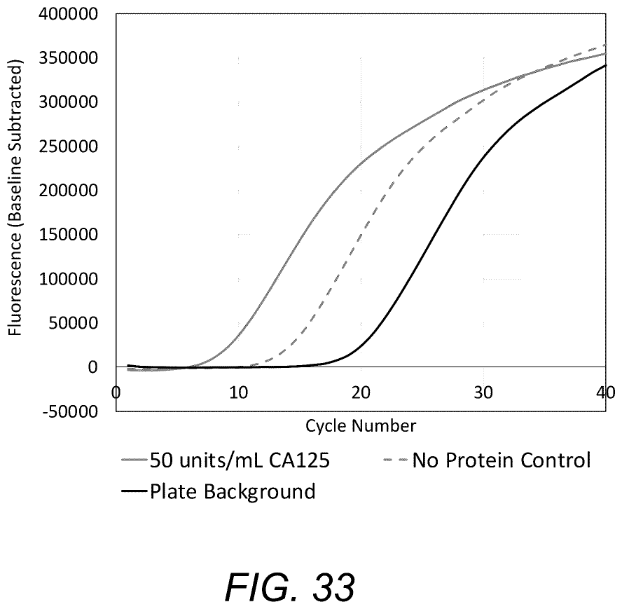

FIG. 33 is a plot showing the quantification of the RCA nanoball product by qPCR. The data show that 50 units/mL of CA-125 target yields about 14-fold more RCA product than the no protein control (2^{3.8}). The modified POC protein assay demonstrates that protein detection by antibodies can be converted to an RCA output.

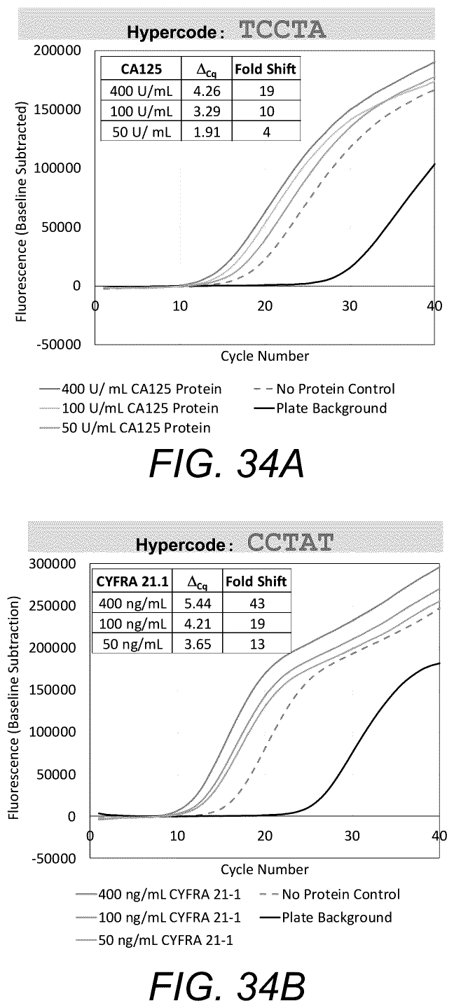

FIG. 34A and FIG. 34B are plots showing the quantification of the POC assay for the two target proteins CA-125 and Cyfra21-1, respectively. The data show that the modified POC protein assay can be used to successfully detect the two protein targets and orthogonal codes.

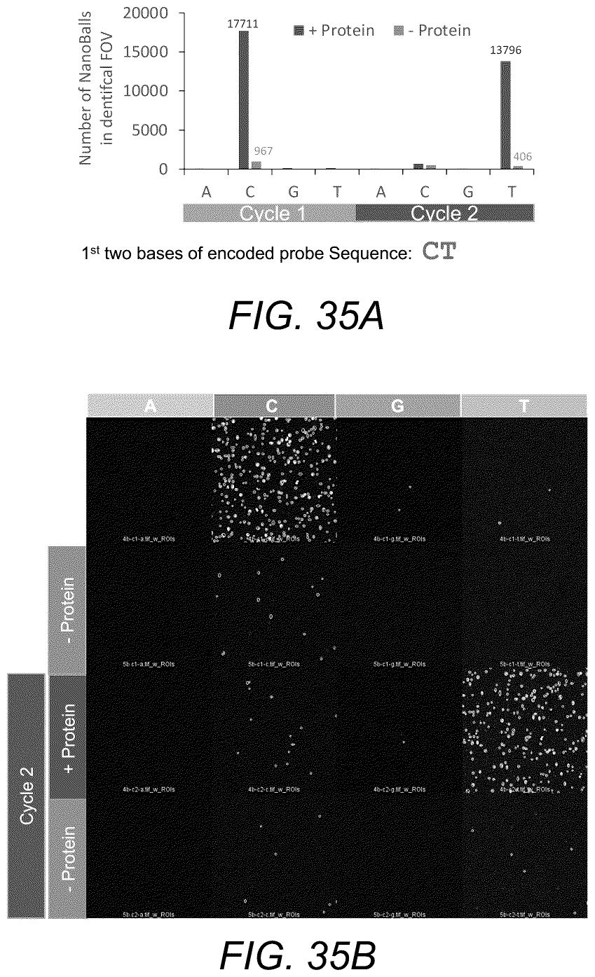

FIG. 35A and FIG. 35B are a plot and a panel of sequencing images for the first 2 bases of the code sequence, respectively. The data show that the modified POC protein assay exhibits low background in sequencing.

### MODEL PROTEIN ASSAY PROTOCOL

### Materials

### Day 1

1) Deionized water
2) 20× wash buffer—warmed to RT
3) Assay diluent
4) Nuclease free water
5) 8 well strip containing bound antibody from ELISA kit
   a. Human Muc16(CA125) ELISA Kit from Invitrogen; #EHMUC16
   b. Human CYFRA 21-1 ELISA Kit frin RayBio; #ELH-CYFRA211
6) Protein standard from ELISA kit

### Day 2

7) Streptavidin
8) Biotin-oligo-APB0013, ordered from IdtDNA
9) Encoded Probe Oligo—APB0015, ordered from IdtDNA
10) Materials for Ligation
11) Antibody-Biotin conjugate from ELISA kit-1 tube
12) 1× diluent (made day 1)
13) Wash buffer (made day 1)
14) RCA materials

### Day 3

15) EDTA—(0.5 M solution, pH 8; Fisher #BP2482-100)

### Protocol

### Day 1

I. Prepare 1x Wash Buffer (from 20× stock in Kit)
   1) Warm wash buffer 20× to RT
   2) Dilute 5 mL of Wash Buffer+95 mL of deionized H2O
   3) Label as 1× wash buffer
   4) Store@4 C and use within 1 month
II. Prepare 1× Diluent (from 5× stock in Kit)
   1) Dilute 5 times before use (2 mL concentrate+8 mL nuclease free water)
III. Dilute Protein Standards form ELISA Kit(s)
   1) Take one of the two provided vials and spin down
   2) Add 400 uL of 1× diluent, pipette up and down=1000 U/mL stock for CA125 and 400 ng/ml for CYFRA 21.1
   3) Prep the desired concentrations according to the excel sheet
IV. Bind protein to ELISA plate
   1) Remove plate from the fridge and take out the desired number of 8 well strips; re-bag the rest of the strips and put back in 4 C fridge
   2) Bind Protein Antigen
      a. Add 100 uL of protein standard to each well, according to the plate map in the excel sheet
      b. Cover wells and incubate for 2 hours at RT with gentle shaking (435 setting on plate shaker)
      c. Put plate in 4 C fridge overnight

### Day 2

VII. Continue with ELISA next day
   1) Remove plate from the fridge and place back on shaker while preparing antibody solution, streptavidin dilutions, biotin-oligo dilutions, and linear encoded probe dilutions as described below
      a. After approximately 30 min (plate should be warmed to RT), discard solution
      b. Wash 4× with 1× wash buffer (300 uL each time)
      c. Blot against paper towel to dry
VIII. Prep Antibody Biotin Conjugate Concentrate (from Kit)—(1× per well)
   1) Take one of the two vials and spin down
   2) Add 100 uL of 1× diluent, pipette up and down to mix
   3) Store concentrate@4 C and use within 5 days
   4) Dilute concentrate (10.6 uL stock+839.4 uL diluent per 8 well strip), mix well
IX. Prepare Streptavidin dilutions (50 pmol per well)
   1) Prepare dilutions of streptavidin according to the excel sheet
X. Prepare Biotin-Oligo dilutions (50 pmol per well)
   1) Prepare dilutions of the biotin oligo (APB00013) according to the excel sheet
XI. Prepare Linear encoded probe dilutions (50 pmol per well)
   1) Prepare dilutions of the linear encoded probe oligo (APB00015) according to the excel sheet
   2) Optional—prepare encoded probe with a dummy blocking oligo to try to passivate the surface so that encoded probe doesn't stick—have tried 3 uM of a dummy 100 mer—see excel sheet
XII. Continue with ELISA plate
   1) Add Antibody-Biotin Conjugate (1× per well)
      a. Add 100 uL of diluted antibody-biotin conjugate to each well
      b. Incubate for 1 hr
      c. Discard solution
      d. Wash 4× with 1× wash buffer (300 uL each time)
      e. Blot against paper towel to dry
   2) Add streptavidin (50 pmol per well)
      a. Add 100 uL of streptavidin to each well according to the plate map
      b. Incubate for 1 hr
      c. Pipet off solution
      d. Wash 4× with 1× wash buffer (300 uL each time)
      e. Blot against paper towel to dry
   3) Add Biotin-oligo tag (50 pmol per well)
      a. Add 100 uL of biotin Oligo (APB00013) dilution to each well according to the plate map
      b. Incubate for 1 hr
      c. Pipet off solution
      d. Wash 4× with 1× wash buffer (300 uL each time)
      e. Blot against paper towel to dry
   4) Add Linear encoded probe DNA (50 pmol per well, with or without a blocking oligo)
      a. Add 100 uL of encoded probe DNA to each well according to the plate map
      b. Incubate for 1 hr
      c. Pipet off solution
      d. Wash 4× with 1× wash buffer (300 uL each time)
      e. Blot against paper towel to dry
   5) Perform RCA
      a. Prepare MasterMix minus Phi29
      b. Add diluent to each well, according to excel sheet
      c. Add RCA Primer APB0007 to each well
      d. Add Phi29 to MasterMix and mix well
      e. Add MasterMix to each well
      f. Shake and incubate at 30 C overnight

### Day 3

6) Quench with EDTA (quenching RCA polymerase to prevent exonuclease activity on subsequent primers)
   a. Add 2 uL of EDTA (0.5 M) to each well
   b. DO NOT WASH
7) Perform qPCR or sequencing, as desired

### ALTERNATIVE PROTOCOL WITH PREMADE CIRCULAR ENCODED PROBE AND STREPTAVIDIN-OLIGO CONJUGATES

### Materials

### Day 1

16) Deionized water
17) 20× wash buffer-warmed to RT
18) Assay diluent
19) Nuclease free water
20) Streptavidin
21) Biotin-oligo-APB0013, ordered from IdtDNA
22) Encoded Probe Oligo-APB0015, ordered from IdtDNA
23) Materials for Ligation and Exonuclease cleavage
24) 8 well strip containing bound antibody from ELISA kit
   a. Human Muc16(CA125) ELISA Kit from Invitrogen; #EHMUC16
   b. Human CYFRA 21-1 ELISA Kit frin RayBio; #ELH-CYFRA211
25) Protein standard from ELISA kit

### Day 2

26) Antibody-Biotin conjugate from ELISA kit-1 tube
27) 1× diluent (made day 1)
28) Wash buffer (made day 1)
29) SA-oligo conjugate (made day 1)
30) Circular encoded probe (made day 1)
31) RCA materials

### Day 3

32) EDTA—(0.5 M solution, pH 8; Fisher #BP2482-100)

### Protocol

### Day 1

I. Prepare 1× Wash Buffer (from 20× stock in Kit)
   5) Warm wash buffer 20× to RT
   6) Dilute 5 mL of Wash Buffer+95 mL of deionized H2O
   7) Label as 1× wash buffer
   8) Store@4 C and use within 1 month
II. Prepare 1× Diluent (from 5× stock in Kit)
   2) Dilute 5 times before use (2 mL concentrate+8 mL nuclease free water)
III. Prepare streptavidin-DNA conjugate
   1) Resuspend biotin oligo tag (APB0013) at 100 uM in nuclease free water (1047 uL)
   2) Weigh out ˜1 mg of streptavidin and resuspend in 1× diluent according to the excel sheet
   3) Spin resuspended streptavidin at 14000 rcf for 4 min, transfer supernatant to a fresh tube and discard any precipitate (aggregate)
   4) Determine concentration of suspended streptavidin using the calculator in the excel sheet
   5) Add biotin-oligo to streptavidin according to the calculator in the excel sheet
   6) Allow to react at room temperature for 2 hrs
   7) Place in 4 C fridge
   8) Note that the new concentration is lower than the starting streptavidin concentration, see excel sheet for calculation
IV. Prepare Circular Encoded Probe
   1) Resuspend Encoded Probe (APB0015) and Splint (APB0013) at 100 uM in nuclease free water
   2) Perform ligation reaction: mix nuclease free water, 10× HiFi Ligase buffer, encoded probe, Splint and HiFi Taq ligase in a PCR tube according to excel calculator—make enough to circularize 10 nmol of encoded probe
   3) Put reaction in a thermocycler according to the following protocol:
      a. Heat at 95 C for 1 min
      b. Reduce heat to 50 C for 30 minutes
      c. Repeat these steps 5 times
   4) Perform exonuclease cleavage reaction: mix nuclease free water, 10× cutsart buffer, Exonuclease I, Exonuclease II, Lamda Exonuclease according to Excel calculator-add 20 uL of this mastermix to each sample
   5) Put reaction in a thermocycler according to the following protocol:
      a. Heat at 37 C for 1 hr
      b. Increase heat to 80 C for 30 min
   6) Clean up reaction mixture with Invitrogen PureLink Quick PCR Purification kit (#K310002):
      a. Follow procedure according to manufacturer's protocol, using “Binding Buffer B2”
   7) Determine concentration of circular encoded probe via nanodrop using extinction coefficient of linear encoded probe provided by IdtDNA, see Excel calculator
V. Dilute Protein Standards
   4) Take one of the two provided vials and spin down
   5) Add 400 uL of 1× diluent, pipette up and down=1000 U/mL stock for CA125 and 400 ng/ml for CYFRA 21.1
   6) Prep the desired concentrations according to the excel sheet
VI. Bind protein to ELISA plate
   3) Remove plate from the fridge and take out the desired number of 8 well strips; re-bag the rest of the strips and put back in 4 C fridge
   4) Bind Protein Antigen
      a. Add 100 uL of protein standard to each well, according to the plate map in the excel sheet
      b. Cover wells and incubate for 2 hours at RT with gentle shaking (
      c. Put plate in 4 C fridge overnight

### Day 2

VII. Continue with ELISA next day
   2) Remove plate from the fridge and place back on shaker while preparing antibody solution, streptavidin-oligo dilutions and circular encoded probe dilutions as described below
      a. After approximately 30 min (plate should be warmed to RT), discard solution
      b. Wash 4× with 1× wash buffer (300 uL each time)
      c. Blot against paper towel to dry
VIII. Prep Antibody Biotin Conjugate Concentrate (from Kit)
   5) Take one of the two vials and spin down
   6) Add 100 uL of 1× diluent, pipette up and down to mix
   7) Store concentrate @4 C and use within 5 days
   8) Dilute concentrate (10.6 uL stock+839.4 uL diluent per 8 well strip), mix well
VII. Prepare Streptavidin-oligo dilutions
   2) Prepare dilutions of the streptavidin-oligo conjugate prepped yesterday according to the excel sheet
VIII. Prepare circular encoded probe dilutions
   3) Prepare dilutions of circular encoded probe that was prepped yesterday according to the excel sheet
IX. Continue with ELISA plate
   8) Add Antibody-Biotin Conjugate
      a. Add 100 uL of diluted antibody-biotin conjugate to each well
      b. Incubate for yeal hr
      c. Discard solution
      d. Wash 4× with 1× wash buffer (300 uL each time)
      e. Blot against paper towel to dry
   9) Add streptavidin-DNA conjugate
      a. Add 100 uL of streptavidin-DNA to each well according to the plate map
      b. Incubate for 1 hr
      c. Pipet off solution
      d. Wash 4× with 1× wash buffer (300 uL each time)
      e. Blot against paper towel to dry
   10) Add Circular Encoded Probe DNA
      a. Add 100 uL of encoded probe DNA to each well according to the plate map
      b. Incubate for 1 hr
      c. Pipet off solution
      d. Wash 4× with 1× wash buffer (300 uL each time)
      e. Blot against paper towel to dry
   11) Perform RCA
      a. Prepare MasterMix minus Phi29
      b. Add diluent to each well, according to excel sheet
      c. Add RCA Primer APB0007 to each well
      d. Add Phi29 to MasterMix and mix well
      e. Add MasterMix to each well
      f. Shake and incubate at 30 C overnight

### Day 3

12) Quench with EDTA (quenching RCA polymerase to prevent exonuclease activity on subsequent primers)
   a. Add 2 uL of EDTA (0.5 M) to each well
   b. DO NOT WASH
13) Perform qPCR or sequencing, as desired

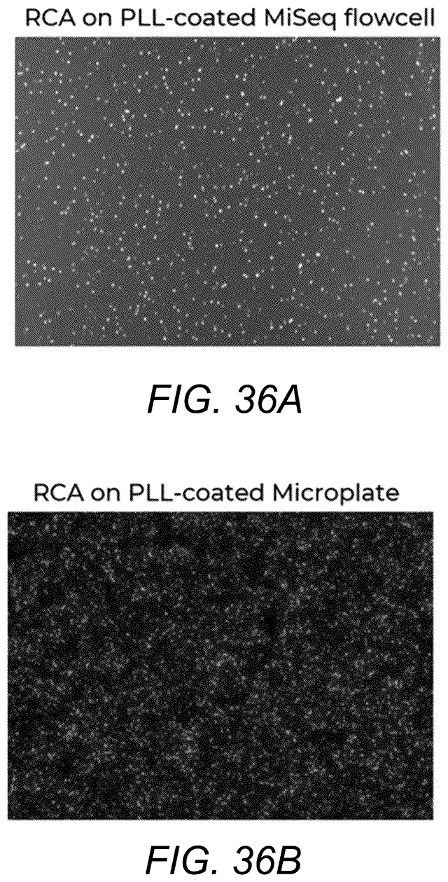

In another experiment, nanoballs were generated in an RCA reaction performed on a polylysine coated surface. Specifically, FIG. 36A and FIG. 36B are photos showing the density, size and uniformity of nanoballs generated in an RCA reaction performed on a polylysine-coated MiSeq flow cell or on a polylysine-coated microplate, respectively. In this example, RCA was performed as follows: RCA on Polylysine surface: MiSeq flowcells were washed to remove surface coatings before 0.01% poly-lysine (PLL) was applied, incubated for 30 minutes, washed and dried. PLL-coated microplates are assembled using purchased PLL-coated glass coverslips and plastic multi-well chambers. RCA reactions are prepared normally in tubes on ice containing phi29 polymerase, buffer, a primer and ligated purified probes, and the complete reaction is applied to the flowcell or microplate. The flowcell or microplate was incubated at 30 C for 6-8 hours, and then washed with Tris/EDTA to stop the reaction. NBs were detected with different methods. The NBs on the MiSeq flowcell were detected by SBS using a MiSeq instrument while the NBs on the microplate surface were hybridized with a fluorophore-labeled oligonucleotide probe and imaged on a Lionheart automated microscope.

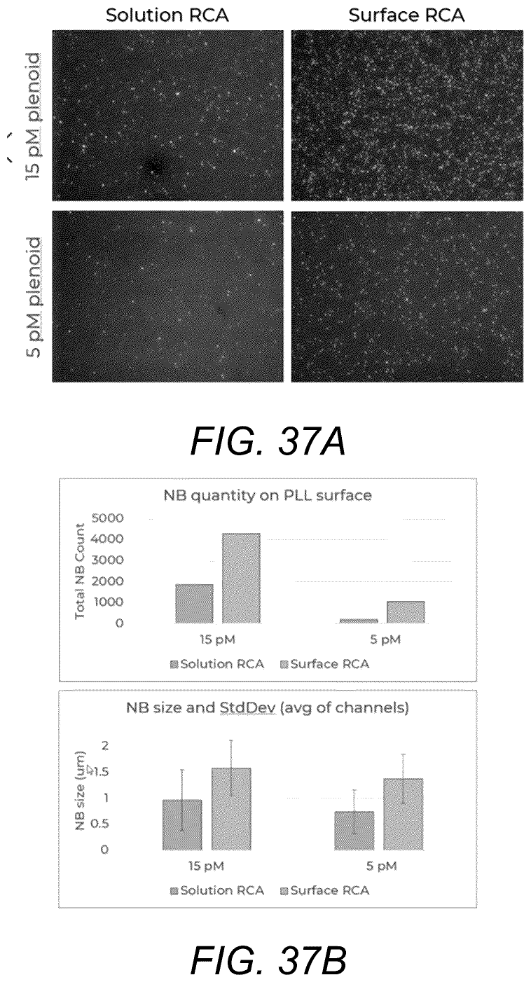

FIG. 37A and 37B are panel of photos and a pair of plots, respectively, of a comparison of nanoballs generated on a polylysine (PLL) surface to nanoballs absorbed to a surface after an RCA solution reaction. In this example, surface vs solution RCA reactions were performed as follows: RCA reactions were prepared normally in tubes on ice containing phi29 polymerase, buffer, a primer and either 5 pM or 15 pM ligated purified probes. A fraction of the RCA reactions was applied to different wells of a microplate with a PLL-coated bottom surface, and then the plate was incubated at 30 C for 4 hours. The remainder of the RCA reactions in tubes were placed at 30 C for 4 hours. The RCA reactions in the microplate were stopped by washing with Tris/EDTA. EDTA and TBS were added to the RCA reactions in tubes and fluorophore-labeled oligonucleotide probes were also added before the reactions were applied to the PLL-coated microplate and allowed to absorb for 1 hr. Fluorophore-labeled oligonucleotide probes in TBS were also applied to the wells in which the RCA was performed in the microplate for specific detection of NBs. After washing, all wells were imaged on a Lionheart automated microscope and analyzed with Lionheart software.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” “comprising,” “include,” “includes,” and “including,” are intended to be non-limiting, such that recitation of items in a list is not to the exclusion of other like items that may be substituted or added to the listed items.

Terms like “preferably,” “commonly,” and “typically” are not utilized herein to limit the scope of the claimed embodiments or to imply that certain features are critical or essential to the structure or function of the claimed embodiments. These terms are intended to highlight alternative or additional features that may or may not be utilized in a particular embodiment of the present disclosure.

The term “substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

Various modifications and variations of the disclosed methods, compositions and uses of the invention will be apparent to the skilled person without departing from the scope and spirit of the invention. Although the invention has been disclosed in connection with specific preferred aspects or embodiments, it should be understood that the invention as claimed should not be unduly limited to such specific aspects or embodiments. The present invention may be implemented using hardware, software, or a combination thereof and may be implemented in one or more computer systems or other processing systems. In one aspect, the invention is directed toward one or more computer systems capable of carrying out the functionality described herein.

In one embodiment, the system includes (a) a reaction vessel; (b) a reagent dispensing module; and (c) software to execute the method of any of the foregoing claims, wherein the method is executed robotically.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

We claim:

1. A method for identifying the presence of polypeptide targets from a sample, comprising:
a) binding a polypeptide target of a set of polypeptide targets from a sample to a capture agent to yield a set of capture agent-target complexes;
b) performing a recognition event on the set of capture agent-target complexes comprising use of a set of encoded probes, wherein:
i) each encoded probe of the set of encoded probes comprises a code from a set of codes, and
ii) each code of the set of codes comprises at least one segment encoding one or more symbols that correspond to a sequence of one or more nucleotides, to yield a set of coded targets each comprising the capture agent-target complex of the set of capture agent-target complexes and an encoded probe of the set of encoded probes;
c) performing a molecular transformation event for each encoded probe of the set of encoded probes to yield a set of modified encoded probes comprising a code of the set of codes in the presence of the polypeptide target and a set of unmodified encoded probes comprising a code of the set of codes in the absence of the polypeptide target, in which the modified encoded probes of the set of modified encoded probes can be amplified and the unmodified encoded probes of the set of unmodified encoded probes cannot be amplified in an amplification event;
d) performing the amplification event for each modified encoded probe of the set of modified encoded probes and detecting the polypeptide target by decoding the codes of the set of codes that are amplified;
wherein decoding the codes comprises recording a signal produced in response to interrogation of each segment of the codes of each amplified and modified encoded probe; and
e) determining a probability of the presence of each of the codes by applying a soft-decision probabilistic decoding algorithm to the recorded signal, wherein the presence of the code is indicative of the presence of the polypeptide target.

2. The method of claim 1, wherein the signal produced is from one or a combination of nanopore sequencing, next generation sequencing, massively parallel sequencing, avidity sequencing, sequencing by synthesis, pyrosequencing, sequencing by hybridization, decoding by hybridization, single molecule real-time sequencing and sequencing by ligation.

3. The method of claim 1, wherein the capture agent comprises two different capture agents, the set of encoded probes comprises split encoded probes, each of the capture agents comprises an oligonucleotide tag complementary to one part of the split encoded probe, and the set of modified encoded probes comprises circularized encoded probes.

4. The method of claim 1, wherein the capture agent comprises two different capture agents, the set of encoded probes comprises split encoded probes, each of the capture agents comprises one part of the split encoded probe, the recognition event comprises introduction of a pair of bridging oligonucleotides, and the set of modified encoded probes comprise circularized encoded probes.

5. The method of claim 1, wherein the capture agent comprises two different capture agents, the set of encoded probes comprise split encoded probes, each of the capture agents comprises one part of the split encoded probe, the recognition event comprises introduction of a single bridging oligonucleotide, and the set of modified encoded probes comprises linear ligated encoded probes.

6. The method of claim 1, wherein the interrogation of the segments comprises decoding by hybridization, and wherein at least one of the segments is interrogated more than one time by hybridization with one or more hybridization probes, wherein each of the one or more hybridization probes comprises at least one different label to produce a signal.

7. The method of claim 6, wherein at least four different labels are utilized in the decoding by hybridization and optionally wherein each code comprises at least four segments and at least sixteen symbols.

8. The method of claim 7, wherein each of the labels comprises a fluorescent label.

9. The method of claim 1, wherein each code comprises one or more of:
a) two or more segments;
b) is a predetermined code sequence;
c) is selected to avoid interaction with other assay components;
d) is selected to ensure that the code differs from each other code from the set of codes, and
e) is homopolymer free.

10. The method of claim 1, wherein each code from the set of codes is generated from one of:
a) a 4-ary nucleotide alphabet of A, C, G and T using a 4-state encoding trellis with three transitions per state; or
b) a 3-ary nucleotide alphabet of a set of three of A, C, G and T using a 4-state encoding trellis with three transitions per state.

11. The method of claim 1, wherein the codes in the set of encoded probes are the same length.

12. The method of claim 1, wherein at least a subset of the set of encoded probes has codes of the same length.

13. The method of claim 1, wherein the set of encoded probes comprises tens, hundreds, thousands, or up to tens of thousands of encoded probes, wherein decoding the codes of the set of codes that are amplified comprises decoding the codes by a soft decision decoding method, and wherein the codes are trellis codes and at least a subset of the trellis codes has the same length.

14. The method of claim 1, wherein each encoded probe in the set of encoded probes is a soft decodable probe.

15. The method of claim 1, wherein the polypeptide targets are extracted from the sample prior to a).

16. The method of claim 1, where the sample comprising polypeptide targets is extracted from whole blood, serum, plasma, or saliva prior to a).

17. The method of claim 1, wherein the sample comprising polypeptide targets is extracted from one or more of a biological fluid, a mammal, a non-mammal, a plant, a sample comprising a virus, and a sample comprising a pathogen prior to a).

18. A composition comprising a set of encoded probes, wherein each encoded probe of the set of encoded probes comprises a code from a set of codes, wherein each code from the set of codes is a soft decodable code comprising a plurality of segments, wherein each segment of the plurality of segments encodes one or more symbols that correspond to a sequence of one or more nucleotides in the segment, and wherein the code from the set of encoded probes is unique for each encoded probe and serves as a proxy for the presence of a polypeptide target.

19. The composition of claim 18, further comprising a target nucleic acid molecule that identifies the polypeptide target, wherein the target nucleic acid molecule is hybridized to at least one encoded probe from the set of encoded probes.

20. The composition of claim 19, further comprises an antibody bound to the target nucleic acid molecule that is hybridized to at least one encoded probe from the set of encoded probes.
